(19) United States
(12) Patent Application Publication
(10) Pub. No.: US 2020/0054571 A1
(43) Pub. Date: Feb. 20, 2020

(54) FORMULATION FOR PROTECTION THROUGH CONTROLLED RELEASE OF MICROPARTICLES CONTAINING RECOMBINANT OUTER MEMBRANE VESICLES

(71) Applicant: CORNELL UNIVERSITY, Ithaca, NY (US)

(72) Inventors: David PUTNAM, Ithaca, NY (US); Hannah C. WATKINS, Ithaca, NY (US); Matthew P. DELISA, Ithaca, NY (US); Gary R. WHITTAKER, Ithaca, NY (US); Cassandra GUARINO, Ithaca, NY (US)

(21) Appl. No.: 16/498,864

(22) PCT Filed: Mar. 29, 2018
(86) PCT No.: PCT/US18/25119

§ 371 Date: Sep. 27, 2019

Related U.S. Application Data
(60) Provisional application No. 62/478,378, filed on Mar. 29, 2017.

(51) Int. Cl. A61K 9/50; A61K 39/385; A61K 39/145; C12N 15/70

(52) U.S. Cl. CPC A61K 9/5021; A61K 39/385; A61K 2039/55555; C12N 15/70; C12N 2760/16134; A61K 39/145

(57) ABSTRACT

The present invention relates to a microparticle. The microparticle includes one or more recombinant outer membrane vesicles, at least some of which display a fusion protein, where the fusion protein comprises at least a portion of a transport protein coupled to at least a portion of one or more antigenic proteins or peptides, and a polymeric coating over the one or more recombinant outer membrane vesicles. The present invention further relates to a method of eliciting an immune response in a mammal and a method of making encapsulated outer membrane vesicles displaying a fusion protein.

This application claims the priority benefit of U.S. Provisional Patent Application Ser. No. 62/478,378 filed Mar. 29, 2017, which is hereby incorporated by reference in its entirety.

This invention was made with government support under Grant Number AI114793 awarded by the National Institutes of Health. The government has certain rights in this invention.

### FIELD OF THE INVENTION

The present invention relates to a formulation for protection through controlled release of microparticles which contain recombinant outer membrane vesicles.

### BACKGROUND OF THE INVENTION

Single dose vaccines offer significant benefits over traditional prime/boost vaccine regimens. Single dose vaccines can increase vaccine population coverage, reduce costs and save time, as patients then require only one healthcare visit. McHugh et al., “Single-Injection Vaccines: Progress, Challenges, and Opportunities,” J. Control. Release 219:596-609 (2015). Additionally, under pandemic conditions, a vaccine that can rapidly induce a protective immune response with a single dose is preferred, yet many vaccines require one or more booster doses to protect the host. There is great interest in single dose vaccine formulations that elicit rapid and long-lasting immune protection.

Poly(lactic-co-glycolic acid) (PLGA), a Food and Drug Administration (FDA) approved biodegradable polymer, is commonly used in drug delivery and is extensively reviewed. Danhier et al., “PLGA-Based Nanoparticles: An Overview of Biomedical Applications,” J. Control. Release 161:505-22 (2012); Mundargi et al., “Nano/micro Technologies for Delivering Macromolecular Therapeutics Using Poly (D, L-lactide-co-glycolide) and its Derivatives,” J. Control. Release 125:193-209 (2008); and Han et al., “Bioerodable PLGA-Based Microparticles for Producing Sustained-Release Drug Formulations and Strategies for Improving Drug Loading,” Front Pharmacol. 7:185 (2016). PLGA microparticles (μP) are commonly used to encapsulate and slowly release small molecules, peptides, and proteins, and are the foundation for a number of products approved by the FDA. Lü et al., “Current Advances in Research and Clinical Applications of PLGA-Based Nanotechnology,” Expert Rev. Mol. Diagn 9:325-41 (2009) and Anselmo et al., “An Overview of Clinical and Commercial Impact of Drug Delivery Systems,” J. Control. Release 190:15-28 (2014). Controlled release vaccine formulations using PLGA μP to encapsulate subunit proteins and adjuvants have had moderate degrees of success, though none are yet commercially available. Silva et al., “PLGA Particulate Delivery Systems for Subunit Vaccines: Linking Particle Properties to Immunogenicity,” Hum. Vaccin. Immunother. 12:1056-69 (2016). In addition to providing a tunable way to control antigen release, PLGA μP can be formulated into sizes that facilitate their uptake by macrophages and dendritic cells, both of which are professional antigen presenting cells. Silva et al., “Poly-(Lactic-Co-Glycolic-Acid)-Based Particulate Vaccines: Particle Uptake by Dendritic Cells is a Key Parameter for Immune Activation,” Vaccine 33:847-54 (2015) and Mao et al., “Effect of WOW Process Parameters on Morphology and Burst Release of FITC-Dextran Loaded PLGA Microspheres,” Int. J. Pharm. 334:137-48 (2007). While PLGA μP have been studied for use in protein subunit—and even DNA—vaccine delivery systems, significantly less work has investigated their ability to release higher order constructs, such as liposomes or other small vesicles. Tinsley-Bown et al., “Formulation of Poly(D,L-Lactic-Co-Glycolic Acid) Microparticles for Rapid Plasmid DNA Delivery,” J. Control. Release 66:229-41 (2000).

Recent reports describe the potential utility of E. coli-derived recombinant outer membrane vesicles (rOMVs) as a safe and effective vaccine approach that directly couples adjuvant with antigen. Rappazzo et al., “Recombinant M2e Outer Membrane Vesicle Vaccines Protect Against Lethal Influenza A Challenge in BALB/c Mice,” Vaccine 34:1252-8 (2016) and Baker et al., “Microbial Biosynthesis of Designer Outer Membrane Vesicles,” Curr. Opin. Biotechnol. 29:76-84 (2014). Transformation of hypervesiculating strains of E. coli with a plasmid that contains a transmembrane protein, cytolysin A (“ClyA”) followed by an antigen of interest, results in the shedding of outer membrane vesicles (diameter: 50-200 nm) that display the antigen of interest. Kim et al., “Engineered Bacterial Outer Membrane Vesicles With Enhanced Functionality,” J. Mol. Biol. 380:51-66 (2008) and Chen et al., “Delivery of Foreign Antigens by Engineered Outer Membrane Vesicle Vaccines,” Proc. Natl. Acad. Sci. USA 107:3099-104 (2010). These rOMVs can then be collected, suspended in buffer, and used as a vaccine, without the need for further protein purification or the addition of supplemental adjuvants. Recently, it was shown that rOMVs that contain peptides derived from the highly conserved matrix 2 protein ectodomain of influenza (“M2e4×Het”) protect against different influenza A subtypes, making M2e4×Het rOMVs a vaccine candidate for protection against pandemic influenza A. Rappazzo et al., “Recombinant M2e Outer Membrane Vesicle Vaccines Protect Against Lethal Influenza A Challenge in BALB/c Mice,” Vaccine 34:1252-8 (2016).

The present invention is directed to overcoming these and other deficiencies in the art.

### SUMMARY OF THE INVENTION

A first aspect of the present invention relates to a microparticle. The microparticle includes one or more recombinant outer membrane vesicles, at least some of which display a fusion protein, where the fusion protein comprises at least a portion of a transport protein coupled to at least a portion of one or more antigenic proteins or peptides, and a polymeric coating over the one or more recombinant outer membrane vesicles.

Another aspect of the present invention relates to a method of eliciting an immune response in a mammal. The method includes providing a microparticle and administering the microparticle to a mammal under conditions effective to elicit the immune response.

Another aspect of the present invention relates to a method of making encapsulated outer membrane vesicles displaying a fusion protein. The method includes providing one or more recombinant outer membrane vesicles, at least some of which display a fusion protein, where the fusion protein comprises at least a portion of a transport protein coupled to at least a portion of one or more antigenic proteins or peptides and applying a polymeric coating over the one or more recombinant outer membrane vesicles.

The influenza A virus undergoes genetic drift and shift, leaving the general population susceptible to emerging pandemic strains, despite seasonal flu vaccination. In the present invention, a single dose influenza vaccine is described that is derived from recombinant outer membrane vesicles (rOMVs) that display a variation of the highly conserved matrix 2 ectodomain (M2e) of the influenza A virus, released over 30 days from poly(lactic-co-glycolide) (PLGA) microparticles. Four weeks post vaccination, BALB/c mice developed high anti-M2e IgG titers that were equivalent to those generated at 8 weeks in a typical prime/boost vaccine regimen. Challenge of mice with a lethal dose of mouse adapted influenza virus PR8 (H1N1) 10 weeks post vaccination resulted in 100% survival for both rOMV single-dose microparticle and prime/boost vaccinated mice. Anti-M2e IgG1 and IgG2a antibody titers were weighted toward IgG1, but splenocytoes isolated from rOMV single-dose microparticle vaccinated mice produced high levels of IFNγ relative to IL-4 in response to stimulation with M2e peptides, supporting a more Th1 biased immune response. The protective immune response was long lasting, eliciting sustained antibody titers and 100% survival of mice challenged with a lethal dose of PR8 six months post initial vaccination. Together, this data demonstrates that rOMVs containing the M2e construct and released from microparticles have potential as single dose vaccine formulations against pandemic influenza, with rapid titer production and long-lasting protection.

In the present invention, using M2e4×Het rOMVs, it was found that 1) rOMVs could be released in a controlled fashion from PLGA μP, 2) the controlled release of rOMVs could lead to immune protection, equivalent to a traditional prime/boost regimen, with a single dose, and 3) there was longevity of a single dose rOMV formulation vs. a traditional prime/boost regimen. The present results show that the controlled release of these rOMV constructs has potential as a single dose vaccine to protect against influenza A challenge, with rapid generation of antibody titers that remain protective for at least six months in mice.

The present invention unexpectedly discovered that the encapsulated rOMVs released from microparticles rapidly produced antibodies with just a single dose while also providing durable immunity in subjects. The data demonstrates that rOMVs containing the construct of the present invention released from microparticles may be used as a single dose vaccine formulation against pandemic influenza, with rapid titer production and long-lasting protection. Based on this result, it is expected that these variations apply regardless of the polymer used in the microparticle and regardless of the antigenic protein or peptide used. Moreover, if the size of the microparticles are the same and the polymer has no adjuvant effects, then the immune response is primarily governed by the rate of release of the rOMVs from the microparticle. Accordingly, the data will extrapolate to all microspheres made from all materials that give the same release kinetics. The rOMVs are inert immunologically when inside the microsphere and the immune response is only induced when the rOMVs, which contain the antigen, are released from the microparticle.

### BRIEF DESCRIPTION OF THE DRAWINGS

FIGS. 1A-1C depict properties of the rOMV-loaded PLGA microparticles. FIG. 1A shows an SEM image of M2e4×Het rOMV loaded PLGA microparticles. FIG. 1B shows an in vitro release profile of M2e4×Het rOMV loaded PLGA microparticles in PBS at 37° C. (n=4 samples). FIG. 1C illustrates the experimental timeline for the present invention.

FIGS. 2A-2C compare anti-M2e IgG titers elicited by PLGA μP and free rOMVs (FIG. 2A), Anti-M2e IgG1 and IgG2a titers at 4 weeks (FIG. 2B) and at 8 weeks (FIG. 2C) post prime. Titers display geomean average (n=15 mice) with 95% confidence intervals (*p<0.05).

FIGS. 3A-3D compare the mortality, morbidity, IFNγ, and IL-4 levels of the PLGA μP and free rOMVs. FIG. 3A shows mortality and FIG. 3B shows morbidity of mice challenged with influenza A/PR8 at 10 weeks post prime vaccination (n=5 mice). Error bars represent standard error of the mean. FIG. 3C shows production of cytokines IFNγ and FIG. 3D shows IL-4 levels 6 days post influenza A/PR8 challenge in PLGA μP, free rOMVs, and PBS vaccinated mice (n=5 mice). Error bars represent standard deviation of average (*p<0.05).

FIGS. 4A-4B compare the PLGA μP and free rOMVs. FIG. 4A depicts anti-M2e IgG titers from week 10 post prime vaccination to week 26. FIG. 4B shows anti-M2e IgG1 and IgG2a titers at 26 weeks post prime vaccination. Error bars represent 95% confidence intervals of geometric mean (n=5 mice, except week 26 group PLGA μP n=4).

FIGS. 5A-5B depict mortality and morbidity of PLGA μP and free rOMVs. FIG. 5A shows mortality and FIG. 5B shows morbidity of mice challenged with influenza A/PR8 at 26 weeks post prime vaccination (n=5 mice, except PLGA μP n=4). Error bars represent standard error of mean (*p<0.05).

### DETAILED DESCRIPTION OF THE INVENTION

A first aspect of the present invention relates to a microparticle. The microparticle includes one or more recombinant outer membrane vesicles, at least some of which display a fusion protein, where the fusion protein comprises at least a portion of a transport protein coupled to at least a portion of one or more antigenic proteins or peptides, and a polymeric coating over the one or more recombinant outer membrane vesicles.

In one embodiment, the microparticle includes a plurality of rOMVs that display a variation of the fusion protein. In one embodiment, the rOMVs display a variation of a highly conserved matrix 2 ectodomain (M2e) of the influenza A virus, which is released over a period of between 1 and 30 days. In one embodiment, the release period of the microparticle is about 10 days, about 20 days, or about 30 days. In a preferred embodiment, the release period is about 30 days.

The microparticle of the present invention can have any suitable shape. For example, the present microparticle and/or its inner core can have a shape of sphere, square, rectangle, triangle, circular disc, cube-like shape, cube, rectangular parallelepiped (cuboid), cone, cylinder, prism, pyramid, right-angled circular cylinder and other regular or irregular shape.

The present microparticle can have any suitable size. For example, the microparticle may have a diameter from about 1 μm to about 800 μm. In certain embodiments, the diameter of the microparticle is about 50 to about 500 μm. In other embodiments, the diameter of the microparticle can be about 50 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, or about 800 μm. In another embodiment, the microparticle may be about 1 μm, about 2 μm, about 3 μm, about 4 μm, about 5 μm, about 6 μm, about 7 μm, about 8 μm, about 9 μm, or about 10 μm. In one embodiment, the microparticle has a diameter of about between 2 μm and 8 μm. In one embodiment, the microparticle has a diameter of 4.22+/−2.8 μm.

In one embodiment of the present invention, the microparticle of the present invention comprises a releasable cargo that can be located in any place inside or on the surface of the microparticle. A trigger for releasing the releasable cargo from the microparticle includes, but is not limited to, contact between the microparticle and a target cell, tissue, organ or subject, or a change of an environmental parameter, such as the pH, ionic condition, temperature, pressure, and other physical or chemical changes, surrounding the microparticle. In certain embodiments, a releasable cargo may comprise one or more therapeutic agents, prophylactic agents, diagnostic or marker agents, or prognostic agents, e.g., an imaging marker, or a combination thereof.

The fusion proteins of the present invention can be generated as described herein or using any other standard technique known in the art. For example, the fusion polypeptide can be prepared by translation of an in-frame fusion of the polynucleotide sequences, i.e., a hybrid gene. The hybrid gene encoding the fusion polypeptide is inserted into an expression vector which is used to transform or transfect a host cell. Alternatively, the polynucleotide sequence encoding the transport protein is inserted into an expression vector in which the polynucleotide encoding the second polypeptide is already present. The second polypeptide or protein of the fusion protein can be fused to the N-, or preferably, to the C-terminal end of the transport protein.

Fusions between the transport protein and an antigenic protein or peptide may be such that the amino acid sequence of the transport protein is directly contiguous with the amino acid sequence of the second protein. Alternatively, the transport protein portion may be coupled to the second protein or polypeptide by way of a linker sequence such as the flexible 5-residue Gly linker described herein or the flexible linkers from an immunoglobulin disclosed in U.S. Pat. No. 5,516,637 to Huang et al, which is hereby incorporated by reference in its entirety. The linker may also contain a protease-specific cleavage site so that the second protein may be controllably released from the transport protein. Examples of protease sites include those specific to cleavage by factor Xa, enterokinase, collagenase, Igase (from Neisseria gonorrhoeae), thrombine, and TEV (Tobacco Etch Virus) protease.

Once the fusion protein is constructed, the nucleic acid construct encoding the protein is inserted into an expression system to which the molecule is heterologous. The heterologous nucleic acid molecule is inserted into the expression system or vector in proper sense (5′→3′) orientation relative to the promoter and any other 5′ regulatory molecules, and correct reading frame. The preparation of the nucleic acid constructs can be carried out using standard cloning methods well known in the art as described by Sambrook et al., Molecular Cloning: A Laboratory Manual, Cold Springs Laboratory Press, Cold Springs Harbor, N.Y. (1989), which is hereby incorporated by reference in its entirety. U.S. Pat. No. 4,237,224 to Cohen and Boyer, which is hereby incorporated by reference in its entirety, also describes the production of expression systems in the form of recombinant plasmids using restriction enzyme cleavage and ligation with DNA ligase.

Suitable expression vectors include those which contain replicon and control sequences that are derived from species compatible with the host cell. For example, if E. coli is used as a host cell, plasmids such as pUC19, pUC18 or pBR322 may be used.

Different genetic signals and processing events control many levels of gene expression (e.g., DNA transcription and messenger RNA (“mRNA”) translation) and subsequently the amount of fusion protein that is displayed on the cell or vesicle surface. Transcription of DNA is dependent upon the presence of a promoter, which is a DNA sequence that directs the binding of RNA polymerase, and thereby promotes mRNA synthesis. Promoters vary in their “strength” (i.e., their ability to promote transcription). For the purposes of expressing a cloned gene, it is desirable to use strong promoters to obtain a high level of transcription and, hence, expression and surface display. Depending upon the host system utilized, any one of a number of suitable promoters may be used. For instance, when using E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV 5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Translation of mRNA in prokaryotes depends upon the presence of the proper prokaryotic signals, which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference in its entirety.

Following transformation of the host cell with an expression vector comprising the nucleic acid construct encoding the fusion protein, the fusion protein is expressed and displayed on the surface of outer membrane vesicles (OMVs).

As used herein, OMV refers to outer membrane vesicles or vesicles, also known as blebs, which are vesicles formed or derived from fragments of the outer membrane of Gram negative or Gram positive bacterium naturally given off during growth. The OMV of the present invention may be recombinantly produced.

As used herein, the term “vesicle” means a hollow particle which may be nano or micro sized. Vesicles carry components encapsulated in the interior, entrapped in the membrane or presented on the surface of the membrane facing outward. Vesicles are formed by an appropriate choice of amphiphilic proteins and/or polypeptides that form the membrane. Some vesicles are formed with single-layer membrane, while others are formed with double-layer membrane.

As used herein, the term recombinant when used in reference to an OMV, cell, nucleic acid, protein, or vector, indicates that the OMV, nucleic acid, protein, or vector, has been modified by the introduction of a heterologous nucleic acid or protein or the alteration of a native nucleic acid or protein, or that the OMV is derived from a cell so modified. Thus, for example, recombinant cells express nucleic acids or polypeptides that are not found within the native (non-recombinant) form of the cell or express native genes that are otherwise abnormally expressed, under expressed, over expressed or not expressed at all. These polypeptides or proteins expressed are also called fusion polypeptides or fusion proteins.

In one embodiment of the present invention, a plurality of proteins or peptides are displayed on the surface of a plurality of rOMVs. The plurality of proteins or peptides displayed on the rOMV are fusion proteins where each fusion protein has a different second protein. The plurality of fusion proteins forms a library of proteins or peptides that are amenable to cell vesicle surface display. In one embodiment, the rOMV is mutated to hyperexpress vesicles containing the fusion protein.

Mutations associated with increased vesicle production are known in the art (McBroom and Kuehn, “Release of Outer Membrane Vesicles by Gram-Negative Bacteria is a Novel Envelope Stress Response,” Mol. Microbiol. 63: 545-558 (2007), which is hereby incorporated by reference in its entirety). For example, disruptions in the nlpl, degS, degP, tolB, pal, rseA, tolA, ponB, tatC, ompR, wzxE, ompC, yieM, pnp, and wag genes have all been shown to result in overproduction of vesicles.

The OMVs or vesicles described herein can be prepared in various ways. Methods for obtaining suitable preparations are disclosed in, for instance, the references cited herein. Techniques for forming OMVs include treating bacteria with a bile acid salt detergent e.g. salts of lithocholic acid, chenodeoxycholic acid, ursodeoxycholic acid, deoxycholic acid, cholic acid, and ursocholic acid. Other techniques may be performed substantially in the absence of detergent using techniques such as sonication, homogenisation, microfluidisation, cavitation, osmotic shock, grinding, French press, and blending, etc (see, e.g., WO2004/019977, which is hereby incorporated by reference in its entirety).

A preferred method for OMV preparation involves ultrafiltration instead of high speed centrifugation on crude OMVs (see, e.g., WO2005/004908, which is hereby incorporated by reference in its entirety). This allows much larger amounts of OMV-containing supernatant to be processed in a much shorter time (typically >15 liters in 4 hours).

In one embodiment, the fusion protein comprises at least a portion of a ClyA protein coupled to at least a portion of one or more antigenic proteins or peptides. Suitable ClyA proteins and nucleic acid molecules encoding them are described below and in U.S. Patent Application Publication No. 2010/0233195 A1 to DeLisa et al., which is hereby incorporated by reference in its entirety.

The present invention further provides that in certain embodiments the rOMVs range in size from about 50 nm to about 200 nm. In certain embodiments, the size of the rOMV is about 50 nm to about 150 nm. In other embodiments, the size of the rOMV can be about 50 nm, about 75 nm, about 100 nm, about 125 nm, about 150 nm, about 175 nm, or about 200 nm. In one embodiment, multiple rOMVs may be contained within a microparticle. Any number of rOMVs may be within a microparticle.

As used herein, a transport protein refers to a protein normally present on the rOMV whose fusion to an antigenic protein or peptide allows display of that antigenic protein or peptide on the surface of the rOMV.

As used herein, the terms protein, peptide, and polypeptide are used interchangeably herein. The conventional one-letter or three-letter code for amino acid residues is used herein. The peptides can be all L-stereo configuration, all D-stereo configuration, or a mixture of L- and D-stereo configuration.

In another embodiment, the transport protein is an adhesin, immunomodulatory compound, protease, or toxin. Examples of such proteins, which have been shown to be associated with bacterial membranes as well as outer membrane vesicles include, without limitation, Apxl, leukotoxin, heat labile enterotoxin, Shiga toxin, ClyA, VacA, OspA and OspD, Haemagglutinin, peptidoglycan hydrolase, phospholipase C, hemolysin, alkaline Phosphatase, Arg-gingipain, Lys gingipain, IpaB, IpaC, IpaD, dentilisin, chitinase, bacteriocin, adhesin, and pore-forming toxin (Keuhn and Kesty, “Bacterial Outer Membrane Vesicles and the Host-Pathogen Interaction,” Genes & Development 19: 2645-2655 (2005), which is hereby incorporated by reference in its entirety). In one embodiment, the transport protein is ClyA.

The antigenic protein or peptide of the present invention may, for example, be any antigenic protein or peptide known in the art, but preferably is derived from pathogenic bacterial organisms, pathogenic fungal organisms, pathogenic viral organisms, parasitic organisms, sexually transmitted disease agents, viral encephalitis agents, protozoan disease agents, fungal disease agents, bacterial disease agents, inflammatory disease agents, autoimmune disease agents, toxic agents, cancer cells, allergens, or combinations thereof.

The antigenic protein or peptide may, for example, be from a pathogenic bacterial organism selected from, but not limited to, the group consisting of Bartonella species, Escherichia species, Bacillus species, Bartonella species, Borrelia species, Bordetella species, Brucella species, Chlamydia species, Clostridium species, Coxiella species, Leptospira species, Neisseria species, Pseudomonas species, Salmonella species, Shigella species, Streptococcus species, Mycobacterium species, Rickettsia species, Treponema species, Vibrio species, Haemophilus species, Enterococcus species, Staphylococcus species, Klebsiella species, Acinetobacter species, Enterobacter species, Moraxella species, Yersinia species, and Francisella species. In one embodiment, the bacterial organism is Mycobacterium tuberculosis.

Antigenic intracellular bacterial proteins or peptides may, for example, be derived from, but not limited to, intracellular pathogens such as Chlamydophila, Ehrlichia, Rickettsia, Mycobacterium, Brucella, Francisella, Legionella, and Listeria. Examples of specific antigenic proteins or peptides include, but are not limited to, the following: Chlamydophila (MOMP, omp2, Cpj0146, Cpj0147, Cpj0308), Ehrlichia (P28 outer membrane protein and hsp60), Mycobacterium (Ag85 complex, MPT32, Phos, Dnak, GroES, MPT46, MPT53, MPT63, ESAT-6 family, MPT59, MAP 85A, MAP 85B, SOD, and MAP 74F), Brucella (BMEII0318, BMEII0513, BME1110748, BMEII1116, BP26, and omp31), Francisella (0-antigen), Legionella (Mip, LPS, outer membrane protein), and Listeria (IspC, lemA, and listeriolysin 0). In one embodiment, the antigenic protein or peptide is 74F protein, which is from Mycobacterium paratuberculosis, the causative agent of Johne's disease in ruminants.

The antigenic protein or peptide may be from a pathogenic fungal organism and may, for example, be selected from, but not limited to, the group consisting of Aspergillus species, Blastomyces species, Candida species, Cryptococcos species, Histoplasma species, Microsporidia species, Mucormycetes species, Pneumocystis species, and Sporothrix species.

The antigenic protein or peptide may be from a viral organism such as, but not limited to, Human Papillomavirus, Alphavirus, Arenavirus, Bunyavirus, Calicivirus, Coronavirus, Enterovirus, Orthomyxovirus, Influenza virus, Hantaanvirus, Reovirus, Flavivirus, Filovirus, Herpes virus, Cytomegalovirus, Varicella-Zoster virus, Epstein-Barr virus, Parvovirus, Paramyxovirus, Polyomavirus, Poxvirus, Rubella virus, Hepatitis virus, Reovirus (Rabies virus), Retrovirus, human immunodeficiency virus (HIV), Norovirus (Norwalk virus), Hemorrhagic fever virus, Mosquito and Tick-borne encephalitis virus, and Prions. The Filovirus may, in certain embodiments, Ebola Virus or Marburg virus.

The antigenic protein or peptide may, for example, be from a parasitic organism such as, but not limited to, Acanthamoeba species, Babesia species, Cryptosporidium species, Entamoeba species, Giardia species, Leishmania species, Naegleria species, Plasmodium species, Toxoplasma species, Trichomonas species, or Trypanosoma species.

In accordance with this and other aspects of the present invention, antigenic viral proteins or peptides may, in some embodiments, be derived from, for example, the following viruses, but not limited to: Human Immunodeficiency Virus (HIV) (p24, gp120, and gp40), influenza A virus (HA and NA), influenza B virus (HA and NA), influenza C virus (HA and NA), rabies virus Glycoprotein G), vesicular stomatitis virus, respiratory syncytial virus, measles virus, parainfluenza virus, mumps virus, yellow fever virus, west nile virus, dengue virus (CPC, MPM, and EPE), rubella virus, sindbis virus, semliki forest virus, ross river virus, rotavirus, parvovirus, JC polyoma virus, BK polyoma virus, Human papillomavirus (HPV), adenovirus, hepatitis B virus, hepatitis C virus (E1 and E2), hepatitis A virus, hepatitis E virus, Human herpesvirus, vaccinia virus, monkeypox virus, cowpox virus, human T-cell leukemia virus, coxsackie virus, polio virus, rhinovirus (VP1-3), enterovirus, echovirus, ebola virus (GP1 and GP2), coronavirus (CoV-N, CoV-S, CoV-M, CoV-E), variola virus, hantaan virus, adeno-associated virus, astrovirus, hendra virus, lassa virus, nipah virus, Marburg virus (NPC1, GP1,2), and Norwalk virus. In one embodiment, the antigenic viral protein or peptide is H1N1 hemagglutinin.

The most common antigenic viral proteins or peptides are derived from food allergy proteins, such as from milk, eggs, fish, crustacean shellfish, tree nuts, peanuts, wheat, coconut, and soybeans. Examples of specific food allergy proteins include, but are not limited to, the following: milk (Bosd4, Bosd5, and Bosd6), eggs (ovomucoid, ovalbumin, ovotransferrin, lysozyme, and alpha-livetin), fish (Gadm1, Gadm2, Gadm3, Sals1, Sals2, Sals3, Gadc1, and Xipg1), crustacean shellfish (Homa1, Homa3, Homa6, Penm1, Penm2, Penm3, Penm4, Penm6, Litv1, Litv2, Litv3, Litv4, and Chan), tree nuts (Prudu3, Prudu4, Prudu5, Prudu6, Jugn1, Jugn2, Jugr1, Jugr2, Bere2, Bere1, Cass5, Cora 1.0401, Cora 1.0402, Cora 1.0403, Cora 1.0404, Coral1, Cora8, Cora9, Anah1, pecan protein albumin 2S, and Litc1), peanuts (Arah1, Arah2, Arah3, Arah4, and Arah5), wheat (Tria12, Tria14, Tria18, and Tria19), coconut (CNP1), and soybeans (Glym1, Glym2, Glym3, Glym4, and Glym5). In one embodiment, the food allergy is to peanuts and the antigenic food allergy protein or peptide is Arah2, which is a protein from peanuts. Allergens may include animal products such as, but not limited to, Fel d 1 (a protein in cats), fur and dander, cockroach calyx, wool, and dust mite excretion. Other allergens include allergens from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides. Likewise, further examples of allergens such as drugs include, for example, penicillin, sulfonamides, salicylates; foods such as celery and celeriac, maize, eggs (typically albumen), fruits; legumes such as, for example, beans, peas, peanuts, and soybeans; as well as other food products such as, but not limited to milk, seafood, sesame, soy, tree nuts, pecans, almonds, and wheat. Other exemplary allergens include, for example, insect stings such as bee sting venom, wasp sting venom, and mosquito stings, as well as mold spores and plant pollens (tree, herb, weed, and grass), ryegrass, timothy-grass, weeds such as ragweed, plantago, nettle, Artemisia vulgaris, Chenopodium album, and sorrel, and trees such as birch, alder, hazel, hornbeam, Aesculus, willow, poplar, Platanus, Tilia, Olea, Ashe juniper, and Alstonia scholaris. Important pollen allergens from trees, grasses and herbs originate from the taxonomic orders of Fagales, Oleales, Pinales and platanaceae including birch (Betula), aider (Alnus), hazel (Corylus), hornbeam (Carpinus) and olive (Olea), cedar (Cryptomeria and Juniperus), Plane tree (Platanus), the order of Poales including i.e. grasses of the genera Lolium, Phleum, Poa, Cynodon, Dactylis, Holcus, Phalaris, Secale, and Sorghum, the orders of Asterales and Urticales including herbs of the genera Ambrosia, Artemisia, and Parietaria. Additional allergens may include latex, wood, Nickel, Chromium, Cadmium, nickel sulfate, balsam of Peru, fragrance, quaternium-15, and neomycin. Still other allergen antigens that may be used include inhalation allergens from fungi such as from the genera Alternaria and Cladosporium.

In one embodiment, the antigenic protein or peptide is a protein or peptide derived from the matrix 2 protein ectodomain of Influenza virus (M2e4×Het or Human influenza A virus M2 protein). Human influenza A virus M2 protein can, for example, be an influenza matrix protein 2 encoded by segment 7 of the influenza A virus genome. Human influenza A virus M2 protein is usually produced by translation from a mRNA derived from this viral genome segment. In some embodiments, M2 usually comprises 97 amino acids. Ectodomain region of human influenza A virus M2 protein or M2e can, for example, relate to the N-terminal externally exposed domain (ectodomain) of Human influenza A virus M2 usually comprising 23 or 24 amino acids (in the 23-mer case the N-terminal methionine is absent). In one embodiment, a peptide obtained or derived from the ectodomain region of human influenza A virus M2 protein comprises a peptide obtained or derived from H1N1, H3N1, H3N2, H5N1, H7N2, as described in Kowalczyk et al., “Strategies and Limitations in Dendrimeric Immunogen Synthesis. The Influenza Virus M2e Epitope As a Case Study,” Bioconjugate Chem. 21:102-110 (2010) and U.S. Patent Publication No. 2012/0058154 to Ilyinskii et al., both of which are hereby incorporated by reference in their entirety.

The polymeric coating of the microparticle of the present invention may be formed from one or more polymers, copolymers, or polymer blends. In some embodiments, the one or more polymers, copolymers, or polymer blends are biodegradable. Examples of suitable polymers include, but are not limited to, a polymer selected from the group consisting of polyesters, polyesteramides, polyamides (including synthetic and natural polyamides), polyphosphazines, polypropyl fumarates, poly(amino acids), polyethers, polyacetals, polycyanoacrylates, polyurethanes, polycarbonates such as tyrosine polycarbonates, polyanhydrides, poly(ortho esters), polyhydroxyacids such as poly(lactic acid), poly(glycolic acid), and poly(lactic acid-co-glycolic acids), polycaprolactone, polyacrylates, polymethacrylates, polyethylene-vinyl acetates, cellulose acetate polymers, polystyrenes, poly(vinyl chloride), poly(vinyl fluoride), poly(vinyl imidazole), poly(vinyl alcohol), water insoluble proteins, crosslinked proteins, aggregated proteins, water insoluble polysaccharides, crosslinked polysaccharides, aggregated polysaccharides, water insoluble polynucleotides, crosslinked polynucleotides, aggregated polynucleotides, water insoluble lipids and adducts thereof, crosslinked lipids and adducts thereof, and aggregated lipids and adducts thereof. In one embodiment, the polymeric coating is a polymer selected from the group consisting of poly(lactic-co-glycolic acid) (PLGA), polycaprolactone, polyglycolide, polylactic acid, and poly-3-hydroxybutyrate. Examples of polymers that may be useful in the microparticles of the present invention further include poly(hydroxyalkanoates); poly(lactide-co-caprolactones); poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), poly(alkylene glycols) such as polyethylene glycol (PEG), poly(propylene glycol) (PPG), and copolymers of ethylene glycol and propylene glycol, poly(oxyethylated polyol), poly(olefinic alcohol), polyvinylpyrrolidone), poly(hydroxy alkylmethacrylamide), poly(hydroxyalkylmethacrylate), poly(saccharides), poly(vinyl alcohol), as well as blends and copolymers thereof. Techniques for preparing suitable polymeric nanoparticles are known in the art, and include solvent evaporation, hot melt particle formation, solvent removal, spray drying, phase inversion, coacervation, and low temperature casting.

In some embodiments, the polymeric coating may be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group).

In some embodiments, the polymeric coating may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides. Certain embodiments may be made using the general teachings of U.S. Pat. No. 5,543,158 to Gref et al. and WO2009/051837 by Von Andrian et al., both of which are hereby incorporated by reference in their entirety.

In some embodiments, the polymeric coating may be modified with a lipid or fatty acid group. A fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In a preferred embodiment, the polyesters may include lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof). In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In a preferred embodiment, the polymeric coating is PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, the polymeric coating can be made of cationic polymers. In general, cationic polymers are able to condense and/or protect negatively charged strands of nucleic acids (e.g. DNA or derivatives thereof).

In some embodiments, the polymeric coating can be degradable polyesters bearing cationic side chains.

The properties of these and other the polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and U.S. Pat. No. 4,946,929; Wang et al., “A Novel Biodegradable Gene Carrier Based on Polyphosphoester,” J. Am. Chem. Soc. 123:9480 (2001); Lim et al., “Cationic Hyperbranched Poly(amino ester): a Novel Class of DNA Condensing Molecule With Cationic Surface, Biodegradable Three-Dimensional Structure, and Tertiary Amine Groups in the Interior,” J. Am. Chem. Soc. 123:2460-1 (2001); Langer, “Biomaterials in Drug Delivery and Tissue Engineering: One Laboratory's Experience,” Acc. Chem. Res. 33:94-101 (2000); Langer et al., “Selected Advances in Drug Delivery and Tissue Engineering,” J. Control. Release 62:7-11 (1999); and Uhrich et al., “Polymeric Systems For Controlled Drug Release,” Chem. Rev. 99:3181-98 (1999), all of which are hereby incorporated by reference in their entirety). More generally, a variety of methods for synthesizing certain suitable polymers are described in The Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., “Facile Synthesis of Block Copolypeptides of Defined Architecture,” Nature 390:386 (1997); and in U.S. Pat. Nos. 6,506,577, 6,632,922, 6,686,446, and 6,818,732, all of which are hereby incorporated by reference in their entirety.

In some embodiments, polymers making up the polymeric coating are linear or branched polymers. In some embodiments, the polymers can be dendrimers. In some embodiments, the polymers can be substantially cross-linked to one another. In some embodiments, the polymers can be substantially free of cross-links. The coating of the microparticle of the present invention may also include block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

The microparticle of the present invention may be administered with one or more free recombinant outer membrane vesicles, at least some of which display a fusion protein, wherein the fusion protein comprises at least a portion of a transport protein coupled to at least a portion of one or more antigenic proteins or peptides.

Another aspect of the present invention is directed to a method of eliciting an immune response in a mammal. The method includes providing the microparticle described above and administering the microparticle to a mammal under conditions effective to elicit the immune response.

In accordance with this and all other aspects of the present invention, the term “immune response” refers to the development in a subject of a humoral and/or a cellular immune response to an antigen present in the composition of interest. A “humoral immune response” refers to an immune response mediated by antibody molecules, while a “cellular immune response” is one mediated by T-lymphocytes and/or other white blood cells. The antigen of interest may also elicit an antibody-mediated immune response. Hence, an immunological response may include one or more of the following effects: the production of antibodies by B-cells; and/or the activation of suppressor, cytotoxic, or helper T-cells and/or T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, which are well known in the art.

The microparticle of the present invention rapidly generates antibody titers that remain protective for at least six months in mice, thereby producing a single dose vaccine with durable immunity. The longevity of the protection afforded by administration of the microparticle, particularly when administered as a single dose rOMV formulation vs. a traditional prime/boost regimen is unique and unexpected. The protection, in one embodiment, lasts at least three months, at least four months, at least five months, at least six months, at least seven months, at least eight months, at least nine months, at least a year, at least two years, at least three years, at least four years, at least five years, at least six years, at least seven years, at least ten years, at least fifteen years, at least twenty years, or at least twenty-five years. In a preferred embodiment, the protection lasts at least 5 years or at least 10 years, with administration of a single dose. The immunogenic compositions can be administered, preferably as a single dose. In one embodiment, high anti-M2e IgG titers indicate that the encapsulated rOMVs in the microparticle elicit a robust humoral response.

In accordance with all aspects of the present invention, a “subject” or “patient” encompasses any animal, but preferably a mammal, e.g., human, non-human primate, a dog, a cat, a horse, a cow, or a rodent. More preferably, the subject or patient is a human. In some embodiments of the present invention, the subject is infected by, or at risk of being infected by, a pathogen. In other embodiments, the subject has, or is at risk of having, a mammalian disease. In further embodiments, the subject has, or is at risk of having, influenza.

A subject at risk of being infected by a pathogen, at risk of having a mammalian disease, or at risk of having influenza may be a subject that has a reduced or suppressed immune system (e.g., due to a disease, condition, or treatment, or a combination thereof). Mammals, such as ruminants, are also at risk due to living in herds. Other at risk subjects may include children, the elderly, as well as hospital workers.

A subject having a food allergy may be selected based upon previous allergy testing methods including skin prick testing, blood tests, and food challenges. Additional diagnostic tools for food allergy include endoscopy, colonoscopy, and biopsy. In a preferred embodiment, the selected subject has a peanut allergy.

The administering of the microparticle is preferably carried out by administration of a single dose. Generally, the amount of the immunogenic compositions that provides an efficacious dose or therapeutically effective dose for vaccination against infection from bacterial, viral, fungal or parasitic infection is from about 1 μg or less to about 100 mg or more, per kg body weight, such as about 1 μg, 2 μg, 5 μg 10 μg 15 μg, 25 μg, 50 μg, 100 μg, 250 μg, 500 μg, 1 mg, 2 mg, 5 mg, 10 mg, 15, mg, 25, mg, 50 mg, or 100 mg per kg body weight.

As used herein, the terms administering of the microparticle of the invention to a mammal is used to prevent, cure, heal, alleviate, relieve, alter, remedy, ameliorate, palliate, improve, prophylactically treat, or affect the mammalian disease, the symptoms of the disease, or the predisposition toward the disease.

As used herein, a “disease” refers to influenza, cardiovascular diseases, inflammatory diseases, cell apoptosis, immune deficiency syndromes, autoimmune diseases, pathogenic infections, cardiovascular and neurological injury, alopecia, aging, Parkinson's disease, Alzheimer's disease, Huntington's disease, acute and chronic neurodegenerative disorders, stroke, vascular dementia, head trauma, ALS, neuromuscular disease, myocardial ischemia, cardiomyopathy, macular degeneration, osteoarthritis, diabetes, acute liver failure, and spinal cord injury. In additional embodiments, other diseases that may be treated include psychiatric disorders which include, but are not limited to, depression, bipolar disorder, and schizophrenia.

Detection of an effective immune response may be determined by a number of assays known in the art. For example, a cell-mediated immunological response can be detected using methods including, lymphoproliferation (lymphocyte activation) assays, CTL cytotoxic cell assays, or by assaying for T-lymphocytes specific for the antigen in a sensitized subject.

Such assays are well known in the art.

The presence of a humoral immunological response can be determined and monitored by testing a biological sample (e.g., blood, plasma, serum, urine, saliva, feces, CSF, or lymph fluid) from the mammal for the presence of antibodies directed to the immunogenic component of the administered product. Methods for detecting antibodies in a biological sample are well known in the art, e.g., ELISA, Dot blots, SDS-PAGE gels or ELISPOT. The presence of a cell-mediated immunological response can be determined by proliferation assays (CD4+ T cells) or CTL (cytotoxic T lymphocyte) assays which are readily known in the art.

In one embodiment of this aspect of the present invention, a microparticle is administered.

Methods for preparing microparticles and cellular vesicles suitable for administration and methods for formulations for administration of microparticles and cellular vesicles are known in the art. Methods of preparing and formulating cellular vesicles, for example, are described above and in U.S. Patent Application Publication No. 2002/0028215 to Kadurugamuwa and Beveridge, WO2006/024946 to Oster et al., and WO2003/051379 to Foster et al., which are hereby incorporated by reference in their entirety. Vesicles may be administered in a convenient manner, such as intravenously, intramuscularly, subcutaneously, intraperitoneally, intranasally, or orally. Preferably the vaccine is administered orally, intramuscularly or subcutaneously. The dosage will depend on the nature of the infection, on the desired effect and on the chosen route of administration, and other factors known to persons skilled in the art.

The microparticles and vesicles of the invention may be administered in a composition with a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances or minor amounts of auxiliary substances such as wetting or emulsifying agents, preservatives or buffers, which enhance the shelf life or effectiveness of the microparticle, rOMVs, protein, or peptide portion may also be used.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies.

The microparticles can be administered using methods known in the art including parenteral, topical, intravenous, oral, subcutaneous, intraperitoneal, intranasal or intramuscular means. The most typical route of administration for compositions formulated to induce an immune response is subcutaneous although others can be equally as effective. The next most common is intramuscular injection. This type of injection is most typically performed in the arm or leg muscles. Intravenous injections as well as intraperitoneal injections, intra-arterial, intracranial, or intradermal injections are also effective in generating an immune response.

In one embodiment, the microparticle may be administered by intravenous infusion or injection. In another embodiment, the microparticle is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., protein or peptide portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The present invention can be administered by a variety of methods known in the art, although for many therapeutic applications, the preferred route/mode of administration is intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems as described above. Biodegradable, biocompatible polymers can be used such as those described above (e.g., ethylene vinyl acetate, polyanhydrides, polyglycolic acid, collagen, polyorthoesters, and polylactic acid). Many methods for the preparation of such formulations are patented or generally known to those skilled in the art. See, e.g., Sustained and Controlled Release Drug Delivery Systems, J. R. Robinson, ed., Marcel Dekker, Inc., New York, 1978, which is hereby incorporated by reference in its entirety.

In certain embodiments, the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

The microparticle of the present invention may be formulated for parenteral administration. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the pharmaceutical agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the agents of the present invention in some embodiments can also be achieved using infusion pump devices such as those described by Medtronic, Northridge, Calif. Such devices allow continuous infusion of desired compounds avoiding multiple injections and multiple manipulations.

In addition to the formulations described previously, the compositions of the present invention may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Effective doses of the microparticle of the present invention, for the induction of an immune response, vary depending upon many different factors, including means of administration, target site, physiological state of the subject, whether the subject is human or an animal, other medications administered, and whether treatment is prophylactic or therapeutic. Treatment dosages need to be titrated to optimize safety and efficacy, and could involve oral treatment.

The transport protein can be an adhesin, immunomodulatory compound, protease, or toxin. Examples of proteins which may be used as transport proteins are described above. Preferably, the transport protein is ClyA.

The antigenic protein or peptide may be derived from and/or selected from the groups of antigenic proteins and peptides described above. Preferably, the antigenic protein or peptide is derived from the matrix 2 protein ectodomain of Influenza virus (M2e4×Het).

The polymeric coating may be a polymer as described above. Preferably, the polymeric coating is PLGA.

The microparticle may be administered with one or more free recombinant outer membrane vesicles, at least some of which display a fusion protein, wherein the fusion protein comprises at least a portion of a transport protein coupled to at least a portion of one or more antigenic proteins or peptides.

The microparticles can be administered in combination with various vaccines either currently being used or in development, whether intended for human or non-human subjects. Examples of vaccines for human subjects and directed to infectious diseases include the combined diphtheria and tetanus toxoids vaccine; pertussis whole cell vaccine; the inactivated influenza vaccine; the 23-valent pneumococcal vaccine; the live measles vaccine; the live mumps vaccine; live rubella vaccine; Bacille Calmette-Guerin I (BCG) tuberculosis vaccine; hepatitis A vaccine; hepatitis B vaccine; hepatitis C vaccine; rabies vaccine (e.g., human diploid cell vaccine); inactivated polio vaccine; meningococcal polysaccharide vaccine; quadrivalent meningococcal conjugate vaccine; yellow fever live virus vaccine; typhoid killed whole cell vaccine; cholera vaccine; Japanese B encephalitis killed virus vaccine; adenovirus vaccine; cytomegalovirus vaccine; rotavirus vaccine; varicella vaccine; anthrax vaccine; small pox vaccine; and other commercially available and experimental vaccines.

Another aspect of the present invention relates to a method of making encapsulated outer membrane vesicles displaying a fusion protein. The method includes providing one or more recombinant outer membrane vesicles, at least some of which display a fusion protein, where the fusion protein comprises at least a portion of a transport protein coupled to at least a portion of one or more antigenic proteins or peptides and applying a polymeric coating over the one or more recombinant outer membrane vesicles.

Encapsulated as described in the present invention can, for example, mean to enclose at least a portion of a substance within the microparticle. In some embodiments, a substance is enclosed completely within a polymer. In other embodiments, most or all of a substance that is encapsulated is not exposed to the local environment external to the microparticle. In other embodiments, no more than 50%, 40%, 30%, 20%, 10% or 5% is exposed to the local environment. Encapsulation is distinct from absorption, which places most or all of a substance on a surface of the microparticle, and leaves the substance exposed to the local environment external to the microparticle. The term encapsulated contemplates any manner by which one or more rOMVs or other material are incorporated, including, for example, attached (by covalent, ionic, or other binding interaction), physical admixture, enveloping the agent in a coating layer, incorporated, distributed throughout the vesicle structure, appended to the surface, encapsulated inside the vesicle, etc.

As used herein, the term coating is, for example, a material and process for making a material where a first substance or substrate surface (e.g., one or more rOMVs) is at least partially covered, fully coated (i.e., encapsulated), or associated with a second substance (e.g., a polymeric coating). In one embodiment, the coating need not be complete or cover the entire surface of the first substance to be coated. The coating may be complete as well (e.g., approximately covering the entire first substance) and form an encapsulation. In some embodiment, there may be multiple coatings and multiple substances within each coating. The coating may vary in thickness or the coating thickness may be substantially uniform. Exemplary compositions of coated particles and methods for coating particles are disclosed in U.S. Pat. No. 6,406,745 to Talton, which is hereby incorporated by reference in its entirety.

A variety of methods of making coatings and encapsulations are well known to those skilled in the art. For example, a double emulsion technique may be used to coat a vesicle with a polymer. Alternatively, encapsulated particles may be prepared by spray-drying. The applying of the polymeric coating over the one or more rOMVs may occur, for example, by a variety of methods as discussed below.

The polymeric coating may be immobilized on the rOMVs using a variety of chemical interactions. For example, a negatively charged PLGA coating can form electrostatic bonds with a second, positively charged coating such as chitosan. This interaction may in certain embodiments prevent the coating from being stripped off the one or more OMVs as it passes into the bloodstream when administered to a subject. In some embodiments, negatively charged coatings may be employed with positively charged cores or, alternatively, positively charged coatings may be used with negatively charged cores. The electrostatic interaction allows for easy fabrication of the particles and facilitates release of the active agent.

Layer-by-layer deposition techniques may be used to coat the particles. For example, vesicles may be suspended in a solution containing the coating material, which then simply adsorbs onto the surface of the vesicles. The coating is not a thick or tight layer but rather allows the active agent to diffuse from the polymer core into the bloodstream when administered to a subject. In addition, the coating may allow enzymes to diffuse from the blood into the vesicle when administered to a subject. Although the coating can remain intact as the vesicle is released, it is itself susceptible to decomposition, and the particle can be fully metabolized.

In addition to electrostatic interactions, other non-covalent interactions may also be used to immobilize a coating. Exemplary non-covalent interactions include but are not limited to affinity interactions, metal coordination, physical adsorption, host-guest interactions, and hydrogen bonding interactions. In one embodiment, the core and the coating may also be linked via covalent interactions.

The transport protein can be an adhesin, immunomodulatory compound, protease, or toxin. Examples of proteins which may be used as transport proteins are described above. Preferably, the transport protein is ClyA.

The antigenic protein or peptide may be derived from and/or selected from the groups of antigenic proteins and peptides described above. Preferably, the antigenic protein or peptide is derived from the matrix 2 protein ectodomain of Influenza virus (M2e4×Het).

The polymeric coating may be a polymer as described above. Preferably, the polymeric coating is PLGA.

In one embodiment, a plurality of fusion proteins are displayed on a plurality of cell vesicles.

### EXAMPLES

The following examples are provided to illustrate embodiments of the present invention but they are by no means intended to limit its scope.

### Example 1—Materials and Methods

M2e-rOMV generation and characterization.

Recombinant OMVs were prepared as previously described. Rappazzo et al., “Recombinant M2e Outer Membrane Vesicle Vaccines Protect Against Lethal Influenza A Challenge in BALB/c Mice,” Vaccine 34:1252-8 (2016) and Rosenthal et al., “Mechanistic Insight Into the Th1-Biased Immune Response to Recombinant Subunit Vaccines Delivered By Probiotic Bacteria-Derived Outer Membrane Vesicles,” PLoS One 9:e112802 (2014), which are hereby incorporated by reference in their entirety. Briefly, E. coli strain ClearColi® ΔnLpI (CC) was transformed with a pBAD plasmid containing transmembrane protein cytolysin A (ClyA) followed by an antigen (M2e4×Het) derived from the ectodomain of the matrix 2 protein (M2e) of influenza A virus. M2e4×Het has previously been expressed and presented on rOMVs and is comprised of four M2e variants separated by glycine-serine linkers and ending in a His-tag. Rappazzo et al., “Recombinant M2e Outer Membrane Vesicle Vaccines Protect Against Lethal Influenza A Challenge in BALB/c Mice,” Vaccine 34:1252-8 (2016), which is hereby incorporated by reference in its entirety. Bacteria were inoculated in terrific broth (TB) (ThermoFisher Scientific, Waltham, U.S.), grown overnight, then sub-cultured to OD600=0.08. When bacteria reached mid-log phase growth, ClyA-M2e4×Het production was induced by addition of L-arabinose to a final concentration of 0.2%. Post induction (18 h), bacteria were centrifuged (5000 rcf, 10 min, 4° C.) and supernatant passed through a 0.2 μm filter. Filtrate was further centrifuged (130,000 rcf, 3 h, 4° C.), the supernatant decanted, the remaining rOMV pellet suspended in sterile phosphate buffered saline (PBS), aliquoted, and stored at −20° C. until use. Total protein concentration in rOMV samples was measured using a Pierce BCA Protein Assay kit according to the manufacturer's instructions (ThermoFisher Scientific, Waltham, U.S.). M2e4×Het content was assessed via Western blot using an anti-His6× primary antibody (Sigma-Aldrich, St. Louis, U.S.).

Formulation of M2e4×Het rOMV loaded PLGA microparticles.

Microparticles loaded with rOMVs were formulated via a water-in-oil-in-water double emulsion (w/o/w). Poly(lactic-co-glycolide) (250 mg, 38-54 kD) with a 50:50 ratio of lactide to glycolide ratio (Sigma-Aldrich, St. Louis, U.S.) was dissolved in dichloromethane (4 mL, DCM) (VWR, Radnor, U.S.). A water-in-oil (w/o) emulsion was then prepared by adding rOmVs (400 μL) at a concentration of 20 mg/mL (surface protein) dropwise to the surface of the DCM/PLGA solution. Emulsification was induced by homogenization at 26,000 rpm with a small sawtooth dispersion head for 30 s (Silverson L5M-A homogenizer). The resulting emulsion was added drop-wise under the liquid surface into 60 mL of a 1.3% polyvinylalcohol (PVA, 31-50 kD, 88% hydrolyzed, Sigma-Aldrich, St. Louis, U.S.) solution while homogenizing with a large dispersion head at a speed of 3000 rpm, followed by an additional 5 minutes of homogenization, to form the double emulsion (w/o/w), The PVA-PLGA-rOMV emulsion was then poured into 200 mL of a 0.3% PVA solution and stirred with a magnetic stirbar uncovered in a fume hood for 7 h to facilitate DCM evaporation and hardening of the microparticles. Subsequently, the PLGA microparticles were washed 3× by centrifugation (4000 rcf, 4° C., 10 min) followed by resuspension each time in 40 mL of sterile water. After the third wash, microparticles were resuspended in 13 mL of sterile water, aliquoted, lyophilized, then stored at −20° C. until use.

Characterization of PLGA microparticles containing rOMVs.

PLGA microparticles (μP) were sputter coated with Au/Pd, then imaged on a Tescan MIRA3 scanning electron microscope (SEM). Total encapsulated rOMV protein was determined by dissolving a known mass of rOMV loaded PLGA microparticles in 0.5 mL of 0.1M NaOH containing 0.5% sodium dodecyl sulfate (SDS) (Sigma-Aldrich, St. Louis, U.S.) and incubating under continuous rotation at 37° C. for 24 hr (n=4 samples). The solution was then neutralized with 0.5 mL 0.1M HCl in PBS and total protein concentration measured using a Pierce BCA Protein Assay (ThermoFisher Scientific, Waltham, U.S.). As a control, unencapsulated rOMVs were treated under the same conditions to account for the influence of the μP dissolution conditions. Total encapsulated rOMV protein was divided by the mass of PLGA μP to determine percent protein encapsulation. Encapsulation efficiency was calculated as percent μP encapsulated protein out of total protein added during μP formulation.

In vitro release profile of rOMV-loaded PLGA microparticles.

The in vitro controlled release profile was generated by suspending particles in 0.5 mL of PBS and incubating at 37° C. under continuous rotation (n=4 samples). Every other day for 30 days and then once weekly, particles were centrifuged (5000 rcf, 20° C., 5 min), then 250 μL of supernatant was collected and replaced by an equal volume of PBS. Protein concentration was quantified using the FluoroProfile Protein Quantification Kit (Sigma-Aldrich, St. Louis, U.S.). The rOMV release time points were stopped upon microparticle degradation (Day 51).

Mouse immunization and study design.

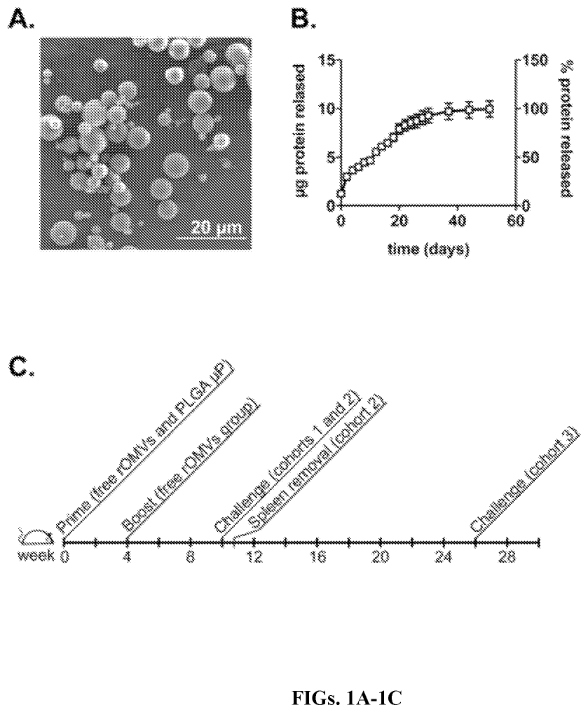

Three groups (n=15 per group) of seven-week-old female BALB/c mice (Jackson Laboratories, Bar Harbor, U.S.) were immunized subcutaneously (s.c.) with 200 μL of each vaccine. The three vaccine regimens were as follows: (1) a single dose of PLGA microparticles loaded with 40 μg of M2e4×Het rOMVs suspended in a PBS solution that contained an additional 40 μg of non-encapsulated (free) M2e4×Het rOMVs (group PLGA pP), (2) a prime dose of 40 μg of free M2e4×Het rOMVs in PBS and a boost dose of the same composition four weeks later (group free rOMVs), and (3) a prime (sham) vaccination of PBS followed by a boost dose of PBS four weeks later (group PBS (sham)). The rOMV preparations contained 5% of total rOMV protein as M2e4×Het (measured by semi-quantitative Western blot), resulting in 40 μg of rOMVs containing ˜2 μg of M2e4×Het antigen. Each of these vaccination groups of 15 mice was further divided into 3 cohorts: cohorts 1 and 2 were challenged at 10 weeks post prime vaccination and cohort 3 was challenged at 26 weeks (six months) post prime vaccination (FIG. 1C). Additionally, mice in cohort 1 that survived the challenge were euthanized at 4 weeks post challenge to end the experiment, mice in cohort 2 were euthanized on day 6 of the challenge to assay spleenocytes, and surviving mice in cohort 3 were euthanized 4 weeks post challenge to end the experiment. Sub-mandibular blood collection was performed at weeks 0, 4, 6, 8, 10, 14, 18, 22, and 26 post prime vaccination. All mouse work was conducted according to protocols approved by Cornell's Institutional Animal Care and Use Committee (IACUC).

Anti-M2e antibody titers (ELISA).

ELISAs to determine anti-M2e IgG, IgG1, and IgG2a titers were performed as previously described. Rappazzo et al., “Recombinant M2e Outer Membrane Vesicle Vaccines Protect Against Lethal Influenza A Challenge in BALB/c Mice,” Vaccine 34:1252-8 (2016), which is hereby incorporated by reference in its entirety. Briefly, 96 well Nunc Maxisorp plates (Thermofisher Scientific, Waltham, U.S.) were coated with M2e peptide (SLLTEVETPIRNEWGCRCNDSSD) (SEQ ID NO: 1) (Lifetein, Hillsborough, U.S.) at 2 μg/mL in PBS and incubated at 37° C. overnight. Plates were washed 2× using wash buffer (PBS with 0.3% bovine serum albumin (BSA) and 0.05% Tween20), then blocked in PBS with 5% milk (Biorad) (20° C., 1 h). Plates were washed 3× with wash buffer, then 2-fold serial dilutions of sera added (n=3 technical replicates, 1 PBS sham control sera sample included per plate) and incubated (37° C., 2 h). Plates were washed 3× with wash buffer, then incubated with appropriate biotin-conjugated secondary antibody (IgG, IgG1, IgG2a) (eBiosciences, San Diego, U.S.) (37° C., 1 h). Plates were washed 3× with wash buffer, then incubated with avidin-horse radish peroxidase (Sigma-Aldrich, St. Louis, U.S.) (37° C., 30 min). Plates were washed 5× with wash buffer, then developed in the dark with TMB (3,3′,5,5′-Tetramethylbenzidine) (20° C., 20 min). Reaction was stopped through addition of 100 μL 4N H2SO4 and absorbance read at OD450 and background absorbance read at OD570. ELISAs were analyzed by first subtracting background OD570 absorbance from OD450 absorbance. Next, the average plus 3 standard deviations of the control sera OD was calculated for each dilution. These control sera values were subtracted from the vaccinated sera samples and the titer was determined as the highest dilution that was still above zero following subtraction.

Influenza challenge.

Mice were challenged with a lethal dose of mouse-adapted H1N1 influenza strain A/Puerto Rico/8/1934 (PR8) (BEI Resources, Manassas, U.S) as previously described. Rappazzo et al., “Recombinant M2e Outer Membrane Vesicle Vaccines Protect Against Lethal Influenza A Challenge in BALB/c Mice,” Vaccine 34:1252-8 (2016), which is hereby incorporated by reference in its entirety. Briefly, PR8 stock was thawed on ice, then diluted to a concentration of 1 fluorescent forming unit (FFU)/μL in sterile PBS. 50 μL of this solution (50 FFU of PR8) was administered intranasally to mice under isoflurane anesthesia. Mice were evaluated for overall health twice daily and weighed once daily to assess response to influenza. Mice were euthanized if weight dropped more than 30% or if they displayed signs of severe distress, as determined by a Board-certified veterinarian.

Cytokine analysis by ELISPOT.

Day 5 post challenge, ELISPOT plates (EMD Millipore, Billerica, U.S.) were coated with anti-IFNγ or anti-IL-4 (R&D Systems, Minneapolis, U.S.) and placed at 4° C. overnight. Day 6 post challenge, mice in cohort 2 were euthanized on day 6 post influenza A/PR8 challenge using CO2 and spleens aseptically removed and placed in complete RPMI media (RPMI media, 10% heat inactivated fetal bovine serum (FBS), 50 U/mL penicillin, 50 U/mL streptomycin) (Thermofisher Scientific, Waltham, U.S.) on ice. Spleens were subsequently mashed using the plunger of a syringe into Petri dishes using 10 mL complete RPMI media, then filtered through a 70 μm sterile screen. Splenocytes were centrifuged (500 rcf, 5 min, 4° C.), then suspended in 1 mL of red blood cell (RBC) lysis buffer (Sigma-Aldrich, St. Louis, U.S.). One minute after addition of RBC lysis buffer, 10 mL of complete RPMI media was added and centrifuged down (500 rcf, 5 min, 4° C.) and washed 2× with complete media. Cells were subsequently diluted to a concentration of 1×106 cells/mL in complete media. The ELISPOT plates were blocked with complete RPMI media for 1 h, then 200 μL of splenoctyes were added per well (5 spleens per cohort, with 3 technical replicates performed from each spleen for each condition). Cells were stimulated with M2e peptide (5 μg/mL), PBS, or cell stimulation cocktail (positive control) (eBiosciences, San Diego, U.S.). Plates were incubated at 37° C. with 5% CO2 for either 24 h (IFNγ) or 48 h (IL-4). Plates were then washed using wash buffer (as described for ELISA) and incubated with anti-IFN or anti-IL-4 (37° C., 1 h). Plates were washed 3× with wash buffer, then incubated with avidin-HRP (37° C., 30 min). Plates were washed 3× with wash buffer and 2× with plain PBS, developed using 3-amino-9-ethylcarbazole (AEC) (BD Biosciences, San Jose, U.S.) monitored in the dark until spots appeared, then the reaction stopped through rinsing wells with tap water. Plates were air dried then sent to ZellNet for reading and spot enumeration (ZellNet Consulting, Inc., Fort Lee, U.S.).

Statistics.

ELISAs were analyzed using n=3 technical replicates per sample per mouse. Titers were averaged using a geometric average and graphed with 95% confidence intervals. IgG titers were compared between the PLGA μP and free rOMVs groups by using a Mann-Whitney test. IgG1:IgG2a titers were compared using a Wilcoxon matched-pairs sign test. Mouse morbidity data was compared using a two-way ANOVA followed by Sidak's test to allow for multiple comparisons between the vaccine groups. Mouse mortality data was analyzed using a log-rank test followed by Bonferoni method of correction between groups. ELISPOT data was analyzed by averaging the technical replicates (n=3) for each spleen, then comparing data from the PLGA μP vaccinated mice spleens and free rOMVs vaccinated mice spleens to the PBS vaccinated mice spleens using an ANOVA followed by using Dunnett's method to allow for multiple comparisons. Statistics were calculated using Graphpad Prism? (La Jolla, U. S).

### Example 2—First Order Release of rOMV from PLGA Microparticles

Poly(lactic-co-glycolide) microparticles (PLGA μP) loaded with M2e-rOMVs were formulated using standard PLGA uP production techniques. The size of rOMV-loaded PLGA uPs was assessed using scanning electron microscopy (SEM); μPs had an average diameter of 4.22+1-2.8 (FIG. 1A). M2e rOMVs range in size from ˜50-200 nm, indicating that multiple rOMVs could be contained within each PLGA μP. Rappazzo et al., “Recombinant M2e Outer Membrane Vesicle Vaccines Protect Against Lethal Influenza A Challenge in BALB/c Mice,” Vaccine 34:1252-8 (2016) and Rosenthal et al., “Mechanistic Insight Into the Th1-Biased Immune Response to Recombinant Subunit Vaccines Delivered By Probiotic Bacteria-Derived Outer Membrane Vesicles,” PLoS One 9:e112802 (2014), which are hereby incorporated by reference in its entirety. Encapsulation efficiency of rOMVs was 37.6%, which is similar to historical values of hydrophilic compounds encapsulated within PlGA using the double emulsion method. PLGA μP contained of 2.18% rOMVs/PLGA mass (w/w). In vitro analysis for rOMV release from PLGA μPs shows a first order release profile that stabilized after 40 days (FIG. 1B). Previously, prime and boost rOMV vaccinations administered 4 weeks apart resulted in the development of high anti-M2e titers and subsequent protection from influenza challenge. Rappazzo et al., “Recombinant M2e Outer Membrane Vesicle Vaccines Protect Against Lethal Influenza A Challenge in BALB/c Mice,” Vaccine 34:1252-8 (2016), which is hereby incorporated by reference in its entirety. Thus, the degradation time period of 40 days-which is likely accelerated in vivo—seemed appropriate for delivery of vaccine. Overall, the ability of rOMV loaded PLGA microparticles to release over a period of several weeks indicated that there was potential for their use as a single dose vaccine. After sixty days, the rOMV remained at the maximum level, providing a basis for extrapolating the extended release to other antigenic proteins and peptides.

### Example 3—Single Dose PLGA rOMV Vaccination Leads to High Anti-M2e Titers

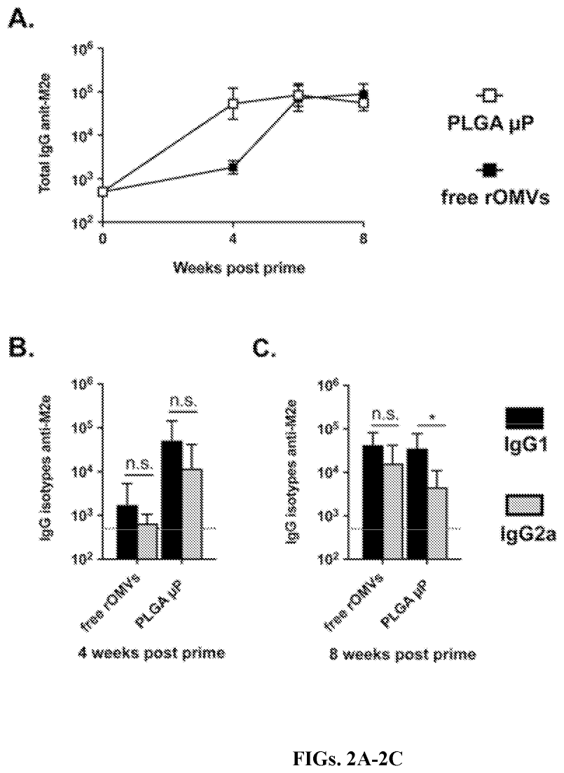

Rapid production of high IgG titers is useful for the creation of a pandemic vaccine, where it is desirable to generate a protective response as quickly as possible. The experimental timeline is represented in FIG. 1C. Mice vaccinated with free rOMVs generated an anti-M2e geometric mean titer of 1,800 four weeks post prime dose, whereas mice vaccinated with rOMV loaded PLGA μPs had a geometric mean titer of 53,200 (FIG. 2A). By six weeks post prime vaccination (and two weeks post boost vaccination of the free rOMVs group) there was no significant difference in anti-M2e IgG levels between the PLGA μP vaccinated group and the free rOMVs group. Titers remained high and remained statistically equivalent at week eight. In addition to total anti-M2e IgG levels, anti-M2e IgG1 and anti-M2e IgG2a levels were also measured. Elevated IgG2a:IgG1 ratios are indicative with a Th1 biased immune response, useful for clearance of viral infections, such as influenza infection. At week 4, both IgG1 and IgG2a anti-M2e titers were barely above those of naïve sera (dotted line) in the free rOMVs group (FIG. 2B). Mice in the PLGA g group had high and statistically equivalent levels of both IgG1 and IgG2a anti-M2e antibodies. By week eight, the free rOMVs group of mice also had high and statistically equivalent IgG1 and IgG2a anti-M2e antibody levels (FIG. 2C). Somewhat surprisingly, at week eight, the PLGA g vaccinated mice had slightly elevated IgG1 titers relative to IgG2a (*p<0.05). Despite this slightly skewed Th2-biased response, the high anti-M2e IgG titers indicated that PLGA encapsulated rOMVs were still capable of eliciting a robust humoral response.

### Example 4—Single Dose PLGA rOMVs Protect BALB/c Mice Against Influenza A Challenge and Elicit a Cellular Response

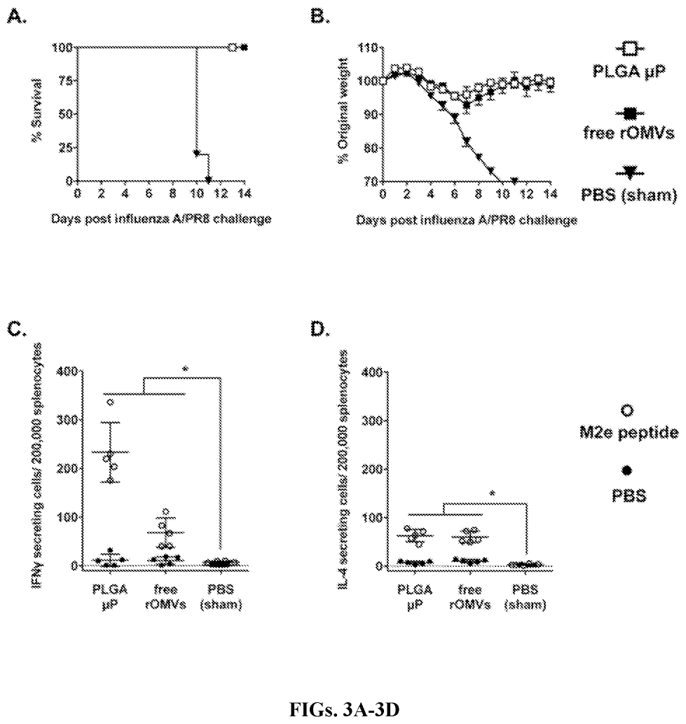

To assess the ability of a PLGA μP rOMV vaccine to protect mice against influenza challenge, mice were exposed to a lethal dose of mouse adapted influenza virus A/Puerto Rico/8/1934 (PR8). PBS vaccinated mice all lost more than 30% of their original body weight, necessitating euthanasia. Both PLGA g vaccinated mice and free rOMVs vaccinated mice had 100% survival following challenge (FIG. 3A). There was no significant difference in weight loss between mice that received the PLGA g vaccine and mice that received the free rOMVs vaccine (FIG. 3B), suggesting that the single dose rOMV vaccine was as effective as the traditional prime/boost rOMV vaccine.

On day six of the challenge, five mice (from cohort 2) from each of the vaccine groups were euthanized and their spleens excised. Splenocytes were subsequently cultured in the presence of M2e peptide or plain PBS and the IFNγ and IL-4 cytokines produced in response to the stimulation analyzed via an ELISPOT assay. IFNγ is associated with a Th1 biased response, whereas IL-4 is associated with a Th2 biased response. Mosmann et al., “TH1 and TH2 Cells: Different Patterns of Lymphokine Secretion Lead to Different Functional Properties,” Annu. Rev. Immunol. 7:145-73 (1989), which is hereby incorporated by reference in its entirety. Splenocytes from both PLGA g and free rOMVs vaccinated mice produced significant levels of IFNγ relative to splenocytes from PBS vaccinated mice when stimulated with M2e peptide (FIG. 3C). Mice vaccinated with PLGA g had especially high levels of IFNγ relative to splenocytes from PBS vaccinated mice, indicating the g were causing a Th1 bias, despite the elevated IgG1:IgG2a anti-M2e antibody ratio at week 8 post injection. Splenocytes from both PLGA and free rOMVS also both produced significantly more IL-4 than PBS vaccinated mice when stimulated with M2e peptide (FIG. 3D). Unlike in IFNγ production, PLGA g and free rOMVs vaccination resulted in similar amounts IL-4 production. The presence of IL-4 as well as IFNγ indicates that the rOMVs generate a fairly balanced Th1/Th2 immune response, which matches the balanced IgG1:IgG2a anti-M2e ratio the free rOMVs vaccinated mouse group displayed. There was no difference in IFNγ or IL-4 production between vaccine groups when splenocytes were treated with plain PBS instead of M2e peptide. Overall, the complete protection elicited by the PLGA μP vaccine indicates that it is a feasible way to formulate a single dose pandemic influenza A vaccine.

### Example 5—Long Term Anti-M2e Titers Result From Both PLGA rOMV Encapsulated and Free rOMVs Vaccination

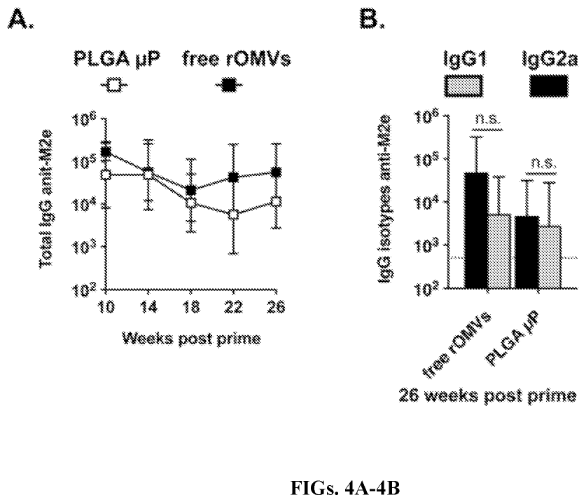

Five mice from each of the vaccine groups were not challenged at week 10 post initial dose; instead, their anti-M2e IgG levels continued to be monitored every four weeks to quantify the level of antibody attrition over time (FIG. 4A). At 10 weeks post prime vaccination, both the PLGA μP and free rOMVs vaccine groups had statistically equivalent anti-M2e IgG titers. The titers remained statistically equivalent over the next 16 weeks, though the average anti-M2e IgG titer was slightly lower in the PLGA μP group than in the free rOMVs group. At 26 weeks post prime vaccination, both the PLGA μP group and free rOMVs group had balanced, statistically equivalent anti-M2e IgG1:IgG2 antibody titers (FIG. 4B). Again, while the geometric averages of the IgG1 and IgG2a anti-M2e titers at 26 weeks were lower than the geometric averages of IgG1 and IgG2a anti-M2e titers at 8 weeks in both the PLGA μP and free rOMVs vaccinated groups, the differences were not statistically significant. The maintenance of these high anti-M2e IgG titers for 26 weeks post the prime vaccination indicates that the PLGA pP and free rOMVs vaccines elicit a long-lasting humoral response.

### Example 6—PLGA rOMV Vaccine Protects BALB/c Mice From Influenza A Challenge Six Months Post Vaccination

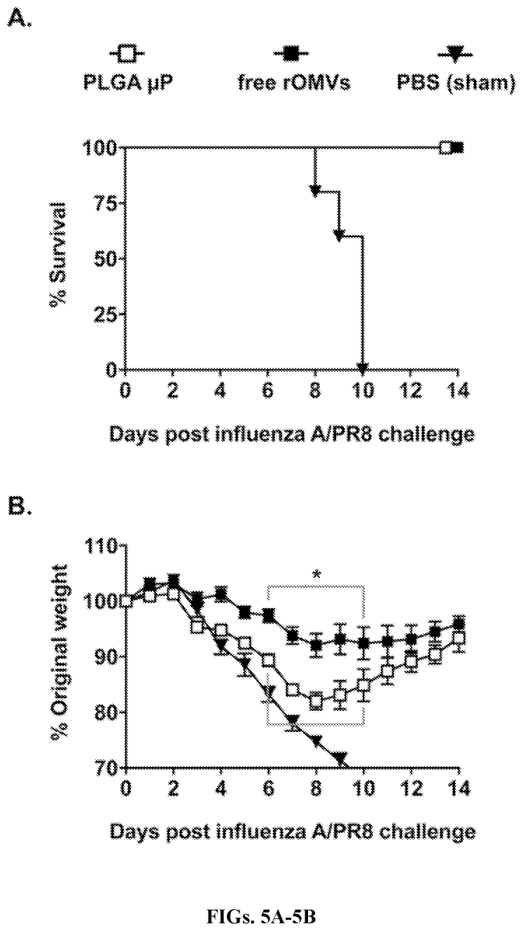

Mice in cohort 3 were challenged with mouse adapted influenza A/PR8 at 26 weeks post their prime vaccination. Equivalent to the challenge that took place at 10 weeks post prime vaccination, mice in the PLGA μP group, free rOMVs group, and PBS (sham) vaccination group all received a lethal dose of PR8. Following challenge, 100% (n=4/4) of PLGA μP vaccinated mice, 100% (n=5/5) of free rOMVs vaccinated mice, and 0% (n=0/5) PBS vaccinated mice survived (FIG. 5A). The number of mice in the PLGA μP cohort was 4 not 5, as one mouse developed a recurring abscess distant from the injection site and required euthanasia at week 24 post prime vaccination. Though both PLGA μP vaccinated mice and free rOMVs vaccinated mice survived, the PLGA μP vaccinated mice experienced significantly more weight loss than the free rOMVs vaccinated mice on days six through ten of challenge. The weight loss experienced by the PLGA g vaccinated mice was still significantly less than that experienced by the PBS (sham) vaccinated mice. Overall, the ability of the PLGA g vaccine to protect mice from challenge six months after it was administered highlights its potential as a single dose vaccine. That the free rOMVs showed such robust challenge protection is also promising, though that vaccine strategy does require administration of both a prime and boost dose.

### Discussion of Examples 2-6

PLGA μP loaded with M2e4×Het rOMVs resulted in effective and long-lasting protection from influenza A/PR8 challenge. Previous work with PLGA g for influenza vaccine development included encapsulated inactivated influenza virus, influenza antigens, and influenza DNA. Hilbert et al., “Biodegradable Microspheres Containing Influenza A Vaccine: Immune Response in Mice,” Vaccine 17:1065-73 (1999); Zhao et al., “Preparation and Immunological Effectiveness of a Swine Influenza DNA Vaccine Encapsulated in PLGA Microspheres,” J. Microencapsul. 27:178-86 (2010); and Raj apaksa et al., “Claudin 4-Targeted Protein Incorporated into PLGA Nanoparticles can Mediate M Cell Targeted Delivery,” J. Control. Release 142:196-205 (2010), which are hereby incorporated by reference in their entirety. The controlled release of rOMV-based vaccines has not been previously reported for any pathogen. Though PLGA g by themselves help to enhance immunogenicity, most require the co-encapsulation of an adjuvant as well as the peptides/protein antigens to generate an immune response. Sharp et al., “Uptake of Particulate Vaccine Adjuvants by Dendritic Cells Activates the NALP3 Inflammasome,” Proc. Natl. Acad. Sci. 106:870-5 (2009) and Oyewumi et al., “Nano-Microparticles as Immune Adjuvants: Correlating Particle Sizes and the Resultant Immune Responses,” Expert Rev. Vaccines 9:1095-107 (2010), which are hereby incorporated by reference in their entirety. Because rOMVs directly couple adjuvant with the antigen, no supplemental adjuvants are necessary. Interestingly, when PLGA g was used to encapsulate inactivated influenza A virus (IAV), it was found that the encapsulated system was less effective than a non-encapsulated IAV vaccine. Singh et al., “Delivery of an Inactivated Avian Influenza Virus Vaccine Adjuvanted with Poly (D, L-Lactic-Co-Glycolic Acid) Encapsulated CpG ODN Induces Protective Immune Responses in Chickens,” Vaccine 34:4807-13 (2016), which is hereby incorporated by reference in its entirety. Instead, it was determined that the best protection from influenza challenge was afforded when just an adjuvant (CpG) loaded PLGA particles were delivered along with IAV, first in a prime intramuscular dose, and then boosted in an intranasal dose. A PLGA-based influenza vaccine system was also developed that encapsulated a cocktail of four conserved influenza A peptides, M2e virus like particles (VLPs), and adjuvant in PLGA nanoparticles (average diameter 260 nm). Hiremath et al., “Entrapment of H1N1 Influenza Virus Derived Conserved Peptides in PLGA Nanoparticles Enhances T Cell Response and Vaccine Efficacy in Pigs,” PLoS One 11:e0151922 (2016), which is hereby incorporated by reference in its entirety. These nanoparticles were delivered to pigs twice intranasally in a prime/boost regimen and resulted in a reduction of symptoms and of viral titers during influenza challenge. While there is some precedent for an M2e-based vaccine delivered with PLGA pP, they required a prime/boost regimen for efficacy.

Previous work also investigated the potential of single-dose influenza vaccine based on M2e, though not through use of PLGA μP. Price et al., “Single-Dose Mucosal Immunization with a Candidate Universal Influenza Vaccine Provides Rapid Protection from Virulent H5N1, H3N2 and H1N1 Viruses,” PLoS One 5:e13162 (2010), which is hereby incorporated by reference its entirety. Conjugation of a short M2e consensus sequence to the papaya mosaic virus was recently used to create a single dose pandemic influenza vaccine. Following a single dose of this vaccine, 70% of BALB/c mice survived lethal influenza challenge and there was as strong positive correlation between IgG2a titers and survival. Carignan et al., “Engineering of the PapMV Vaccine Platform with a Shortened M2e Peptide Leads to an Effective One Dose Influenza Vaccine,” Vaccine 33:7245-53 (2015), which is hereby incorporated by reference in its entirety. While the rOMV-based vaccine reported herein did not elicit elevated IgG2a:IgG1 anti-M2e antibody titers, the survival results show that sufficient IgG2a antibody was present to impart protection from influenza challenge. Steric hindrance from hemagglutinin and neuraminidase typically prevents anti-M2e antibodies from neutralizing virions; therefore, it is beneficial to have high levels of anti-M2e IgG2a antibodies, which help to clear influenza infected cells via antibody dependent cellular cytotoxicity. El Bakkouri et al., “Universal Vaccine Based on Ectodomain of Matrix Protein 2 of Influenza A: Fc Receptors and Alveolar Macrophages Mediate Protection,” J. Immunol. 186:1022-31 (2011), which is hereby incorporated by reference in its entirety.

The rapid development of anti-M2e IgG titers by the PLGA μp vaccine was likely a result of free rOMVs co-administered with the PLGA g formulation. Substantial efforts have gone into characterizing the degradation of PLGA g in tissues and characterizing the release profiles that results from PLGA of varying compositions and sizes. Anderson et al., “Biodegradation and Biocompatibility of PLA and PLGA Microspheres,” Adv. Drug Deliv. Rev. 64:72-82 (2012), which is hereby incorporated by reference in its entirety. Additionally, other systems have explored the delivery of antigen in a controlled release manner that mimics natural infection or a prime/boost dosing regimen. DeMuth et al., “Implantable Silk Composite Microneedles for Programmable Vaccine Release Kinetics and Enhanced Immunogenicity in Transcutaneous Immunization,” Adv. Healthc. Mater. 3:47-58 (2014), which is hereby incorporated by reference in its entirety. However, the present invention found that the simple approach of suspending PLGA μg containing M2e-rOMVs directly in a free rOMV solution led to titers that were equivalent to those elicited with a prime/boost regimen.

The ability of the PLGA μP and free rOMVs vaccines to remain protective 6 months post prime vaccination highlights the translational potential of the platform. Few long-term vaccination studies, complete with challenge, have been conducted using PLGA formulations. Previously, a single dose hepatitis vaccine using PLGA g was evaluated and found to elicit anti-HBsAg antibodies for 125 days (˜4 months) post vaccination, though no challenge studies were performed. Feng et al., “Pharmaceutical and Immunological Evaluation of a Single-Dose Hepatitis B Vaccine Using PLGA Microspheres,” J. Control. Release 112:35-42 (2006), which is hereby incorporated by reference in its entirety. M2e vaccines in particular have been hampered by offering only short term protection—the M2e-based influenza vaccine ACAM-FLU-ATM entered Phase I clinical trials and resulted in high seroconversion rates, but antibody titers quickly dropped, leading to cancellation of Phase II efficacy trials. Deng et al., “M2e-Based Universal Influenza A Vaccines,” Vaccines 3 (2015), which is hereby incorporated by reference in its entirety. While the PLGA g vaccine described in this invention maintained protective antibody titers for 6 months post vaccination, the mice in the PLGA g group did show increased morbidity in the 26-week challenge vs. the challenge that took place at 10 weeks. However, the life expectancy of a laboratory mouse is only about two years, making the protection at 6 months post prime vaccination observed using these rOMV formulations a significant portion of its lifespan. Goodrick et al., “Life-Span and the Inheritance of Longevity of Inbred Mice,” J. Gerontol. 30:257-63 (1975), which is hereby incorporated by reference in its entirety. Some subunit vaccines, such as Gardasil for cervical cancer, produce titers that drop over time, but then remain stable for years, maintaining protection (in humans). Schiller et al., “Raising Expectations for Subunit Vaccine,” J. Infect. Dis. 211:1373-5 (2015), which is hereby incorporated by reference by its entirety. Further work with aged mice rather than 7-week-old mice, as well as other animal models will give a clearer and more nuanced view of the longevity of rOMV-based vaccines.

The splenocytes of mice vaccinated with both PLGA g and free rOMVs produced IFNγ and IL-4 in response to M2e peptide stimulation, indicating a balanced cellular response, as IFNγ is associated with a Th1 biased response and IL-4 with a Th2 biased response. Interestingly, PLGA g vaccinated mice produced significantly more IFNγ in response to M2e peptide stimulation than splenocytes from free rOMVs vaccinated mice during the challenge that took place 10 weeks post prime vaccination. The PLGA g vaccinated mice lost more weight following the second challenge than the first, indicating that the protective response required additional time to clear the virus. Average anti-M2e IgG titers were statistically equivalent at the 10 and 26-week time point, but trended lower at the later time points. Previous work showed that encapsulation of antigens and adjuvants in PLGA can lead to increased cellular response, due in part to enhanced antigen presentation through uptake by macrophages and dendritic cells. Luzardo-Alvarez et al., “Biodegradable Microspheres Alone do not Stimulate Murine Macrophages in Vitro, but Prolong Antigen Presentation by Macrophages in Vitro and Stimulate a Solid Immune Response in Mice,” J. Control. Release 109:62-76 (2005), which is hereby incorporated by reference in its entirety. Previous work showed that dendritic cells engulfed an average of three particles when they were 8 μm in size and one particle when they were 11 μm in size. Rubsamen et al., “Eliciting Cytotoxic T-lymphocyte Responses from Synthetic Vectors Containing One or Two Epitopes in a C57BL/6 Mouse Model Using Peptide-Containing Biodegradable Microspheres and Adjuvants,” Vaccine 32:4111-6 (2014), which is hereby incorporated by reference in its entirety. Additionally, other researchers have added dendritic cell targeting moieties to microparticle formulations in an attempt to enhance cellular uptake. Cruz et al., “Targeting Nanoparticles to CD40, DEC-205 or CD11c Molecules on Dendritic Cells for Efficient CD8+ T Cell Response: A Comparative Study,” J. Control. Release 192:209-18 (2014) and Herrmann et al., “Cytotoxic T Cell Vaccination with PLGA Microspheres Interferes with Influenza A Virus Replication in the Lung and Suppresses the Infectious Disease,” J. Control. Release 216:121-31 (2015), which are hereby incorporated by reference in their entirety. Further work with rOMVs within PLGA g is needed to determine the ideal PLGA size for optimizing potent—and long lasting—cellular responses.

In conclusion, vaccination of BALB/c mice with a single dose of an influenza A vaccine, comprised of M2e4×Het rOMVs loaded into PLGA g and suspended in an M2e4×Het rOMV solution, resulted in 100% survival following lethal influenza A/PR8 challenges at 10 weeks and 26 weeks post prime vaccination. The protective response is likely a combination of cellular and humoral contributions. Following the 10-week challenge, splenocytes from both the PLGA μP vaccinated mice and free rOMVs vaccinated mice responded strongly to the M2e peptide, producing IFNγ and IL-4. Additionally, while the average anti-M2e IgG titers trended lower from 10 weeks to 26 weeks post prime vaccination, the difference was not significant and the protection to influenza challenge was maintained. Overall, the results support the premise that PLGA g represent an innovative way to generate single dose rOMV vaccines.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A microparticle comprising:
one or more recombinant outer membrane vesicles, at least some of which display a fusion protein, wherein said fusion protein comprises at least a portion of a transport protein coupled to at least a portion of one or more antigenic proteins or peptides, and
a polymeric coating over said one or more recombinant outer membrane vesicles.

2. The microparticle of claim 1, wherein the transport protein is an adhesin, immunomodulatory compound, protease, or toxin.

3. The microparticle of claim 1, wherein the transport protein is ClyA.

4. The microparticle of claim 1, wherein the antigenic protein or peptide is a protein or peptide derived from pathogenic bacterial organisms, pathogenic fungal organisms, pathogenic viral organisms, parasitic organisms, sexually transmitted disease agents, viral encephalitis agents, protozoan disease agents, fungal disease agents, bacterial disease agents, inflammatory disease agents, autoimmune disease agents, toxic agents, cancer cells, allergens, and combinations thereof.

5. The microparticle of claim 4, wherein the antigenic protein or peptide is from a pathogenic bacterial organism selected from the group consisting of Bartonella species, Escherichia species, Bacillus species, Bartonella species, Borrelia species, Bordetella species, Brucella species, Chlamydia species, Clostridium species, Coxiella species, Leptospira species, Neisseria species, Pseudomonas species, Salmonella species, Shigella species, Streptococcus species, Mycobacterium species, Rickettsia species, Treponema species, Vibrio species, Haemophilus species, Enterococcus species, Staphylococcus species, Klebsiella species, Acinetobacter species, Enterobacter species, Moraxella species, Francisella species, and Yersinia species.

6. The microparticle of claim 4, wherein the antigenic protein or peptide is from a pathogenic fungal organism selected from the group consisting of Aspergillus species, Blastomyces species, Candida species, Cryptococcos species, Histoplasma species, Microsporidia species, Mucormycetes species, Pneumocystis species, and Sporothrix species.

7. The microparticle of claim 4, wherein the antigenic protein or peptide is from a viral organism selected from the group consisting of Human Papillomavirus, Alphavirus, Arenavirus, Bunyavirus, Calicivirus, Coronavirus, Enterovirus, Orthomyxovirus, Influenza virus, Hantaanvirus, Reovirus, Flavivirus, Filovirus, Herpes virus, Cytomegalovirus, Varicella-Zoster virus, Epstein-Barr virus, Parvovirus, Paramyxovirus, Polyomavirus, Poxvirus, Rubella virus, Hepatitis virus, Reovirus (Rabies virus), Retrovirus, human immunodeficiency virus (HIV), Norovirus (Norwalk virus), Hemorrhagic fever virus, Mosquito and Tick-borne encephalitis virus, and Prions.

8. The microparticle of claim 7, wherein the antigenic protein or peptide is from a Filovirus selected from the group consisting of Ebola Virus and Marburg virus.

9. The microparticle of claim 4, wherein the antigenic protein or peptide is from a parasitic organism selected from the group consisting of Acanthamoeba species, Babesia species, Cryptosporidium species, Entamoeba species, Giardia species, Leishmania species, Naegleria species, Plasmodium species, Toxoplasma species, Trichomonas species, and Trypanosoma species.

10. The microparticle of claim 4, wherein the antigenic protein or peptide is a protein or peptide derived from the matrix 2 protein ectodomain of Influenza virus (M2e4×Het).

11. The microparticle of claim 1, wherein the polymeric coating is a polymer selected from the group consisting of polyesters, polyamides, polyphosphazines, polypropyl fumarates, poly(amino acids), polyethers, polyacetals, polycyanoacrylates, polyurethanes, polycarbonates, polyanhydrides, poly(ortho esters), polyhydroxy acids, polyacrylates, polymethacrylates, polyethylene-vinyl acetates, cellulose acetate polymers, polystyrenes, poly(vinyl chloride), poly(vinyl fluoride), poly(vinyl imidazole), poly(vinyl alcohol), water insoluble proteins, crosslinked proteins, aggregated proteins, water insoluble polysaccharides, crosslinked polysaccharides, aggregated polysaccharides, water insoluble polynucleotides, crosslinked polynucleotides, aggregated polynucleotides, water insoluble lipids and adducts thereof, crosslinked lipids and adducts thereof, and aggregated lipids and adducts thereof.

12. The microparticle of claim 11, wherein the polymeric coating is a polymer selected from the group consisting of poly(lactic-co-glycolic acid) (PLGA), polycaprolactone, polyglycolide, polylactic acid, and poly-3-hydroxybutyrate.

13. The microparticle of claim 12, wherein the polymeric coating is PLGA.

14. A method of eliciting an immune response in a mammal, said method comprising:
providing the microparticle of claim 1 and
administering the microparticle to a mammal under conditions effective to elicit the immune response.

15. The method according to claim 14, wherein the transport protein is an adhesin, immunomodulatory compound, protease, or toxin.

16. The method according to claim 14, wherein the transport protein is ClyA.

17. The method according to claim 14, wherein the antigenic protein or peptide is a protein or peptide derived from pathogenic bacterial organisms, pathogenic fungal organisms, pathogenic viral organisms, parasitic organisms, sexually transmitted disease agents, viral encephalitis agents, protozoan disease agents, fungal disease agents, bacterial disease agents, inflammatory disease agents, autoimmune disease agents, toxic agents, cancer cells, allergens, and combinations thereof.

18. The method according to claim 17, wherein the antigenic protein or peptide is from a pathogenic bacterial organism selected from the group consisting of Bartonella species, Escherichia species, Bacillus species, Bartonella species, Borrelia species, Bordetella species, Brucella species, Chlamydia species, Clostridium species, Coxiella species, Leptospira species, Neisseria species, Pseudomonas species, Salmonella species, Shigella species, Streptococcus species, Mycobacterium species, Rickettsia species, Treponema species, Vibrio species, Haemophilus species, Enterococcus species, Staphylococcus species, Klebsiella species, Acinetobacter species, Enterobacter species, Moraxella species, Francisella species, and Yersinia species.

19. The method according to claim 17, wherein the antigenic protein or peptide is from a pathogenic fungal organism selected from the group consisting of Aspergillus species, Blastomyces species, Candida species, Cryptococcos species, Histoplasma species, Microsporidia species, Mucormycetes species, Pneumocystis species, and Sporothrix species.

20. The method according to claim 17, wherein the antigenic protein or peptide is from a viral organism selected from the group consisting of Human Papillomavirus, Alphavirus, Arenavirus, Bunyavirus, Calicivirus, Coronavirus, Enterovirus, Orthomyxovirus, Influenza virus, Hantaanvirus, Reovirus, Flavivirus, Filovirus, Herpes virus, Cytomegalovirus, Varicella-Zoster virus, Epstein-Barr virus, Parvovirus, Paramyxovirus, Polyomavirus, Poxvirus, Rubella virus, Hepatitis virus, Reovirus (Rabies virus), Retrovirus, human immunodeficiency virus (HIV), Norovirus (Norwalk virus), Hemorrhagic fever virus, Mosquito and Tick-borne encephalitis virus, and Prions.

21. The method according to claim 20, wherein the antigenic protein or peptide is from a Filovirus selected from the group consisting of Ebola Virus and Marburg virus.

22. The method according to claim 17, wherein the antigenic protein or peptide is from a parasitic organism selected from the group consisting of Acanthamoeba species, Babesia species, Cryptosporidium species, Entamoeba species, Giardia species, Leishmania species, Naegleria species, Plasmodium species, Toxoplasma species, Trichomonas species, and Trypanosoma species.

23. The method of claim 17, wherein the antigenic protein or peptide is a protein or peptide derived from the matrix 2 protein ectodomain of Influenza virus (M2e4×Het).

24. The method according to claim 14, wherein the polymeric coating is a polymer selected from the group consisting of polyesters, polyamides, polyphosphazines, polypropyl fumarates, poly(amino acids), polyethers, polyacetals, polycyanoacrylates, polyurethanes, polycarbonates, polyanhydrides, poly(ortho esters), polyhydroxy acids, polyacrylates, polymethacrylates, polyethylene-vinyl acetates, cellulose acetate polymers, polystyrenes, poly(vinyl chloride), poly(vinyl fluoride), poly(vinyl imidazole), poly(vinyl alcohol), water insoluble proteins, crosslinked proteins, aggregated proteins, water insoluble polysaccharides, crosslinked polysaccharides, aggregated polysaccharides, water insoluble polynucleotides, crosslinked polynucleotides, aggregated polynucleotides, water insoluble lipids and adducts thereof, crosslinked lipids and adducts thereof, and aggregated lipids and adducts thereof.

25. The method according to claim 24, wherein the polymeric coating is a polymer selected from the group consisting of poly(lactic-co-glycolic acid) (PLGA), polycaprolactone, polyglycolide, polylactic acid, and poly-3-hydroxybutyrate.

26. The method according to claim 25, wherein the polymeric coating is PLGA.

27. The method according to claim 14, wherein the microparticle is administered with one or more free recombinant outer membrane vesicles, at least some of which display a fusion protein, wherein the fusion protein comprises at least a portion of a transport protein coupled to at least a portion of one or more antigenic proteins or peptides.

28. The method according to claim 14, wherein said administering of the microparticle is carried out by administration of a single dose.

29. A method of making encapsulated outer membrane vesicles displaying a fusion protein, said method comprising:
providing one or more recombinant outer membrane vesicles, at least some of which display a fusion protein, wherein said fusion protein comprises at least a portion of a transport protein coupled to at least a portion of one or more antigenic proteins or peptides and
applying a polymeric coating over said one or more recombinant outer membrane vesicles.

30. The method according to claim 29, wherein the transport protein is an adhesin, immunomodulatory compound, protease, or toxin.

31. The method according to 29, wherein the transport protein is ClyA.

32. The method according to claim 29, wherein the antigenic protein or peptide is a protein or peptide derived from pathogenic bacterial organisms, pathogenic fungal organisms, pathogenic viral organisms, parasitic organisms, sexually transmitted disease agents, viral encephalitis agents, protozoan disease agents, fungal disease agents, bacterial disease agents, inflammatory disease agents, autoimmune disease agents, toxic agents, cancer cells, allergens, and combinations thereof.

33. The method according to claim 32, wherein the antigenic protein or peptide is from a pathogenic bacterial organism selected from the group consisting of Bartonella species, Escherichia species, Bacillus species, Bartonella species, Borrelia species, Bordetella species, Brucella species, Chlamydia species, Clostridium species, Coxiella species, Leptospira species, Neisseria species, Pseudomonas species, Salmonella species, Shigella species, Streptococcus species, Mycobacterium species, Rickettsia species, Treponema species, Vibrio species, Haemophilus species, Enterococcus species, Staphylococcus species, Klebsiella species, Acinetobacter species, Enterobacter species, Moraxella species, Francisella species, and Yersinia species.

34. The method according to claim 32, wherein the antigenic protein or peptide is from a pathogenic fungal organism selected from the group consisting of Aspergillus species, Blastomyces species, Candida species, Cryptococcos species, Histoplasma species, Microsporidia species, Mucormycetes species, Pneumocystis species, and Sporothrix species.

35. The method according to claim 32, wherein the antigenic protein or peptide is from a viral organism selected from the group consisting of Human Papillomavirus, Alphavirus, Arenavirus, Bunyavirus, Calicivirus, Coronavirus, Enterovirus, Orthomyxovirus, Influenza virus, Hantaanvirus, Reovirus, Flavivirus, Filovirus, Herpes virus, Cytomegalovirus, Varicella-Zoster virus, Epstein-Barr virus, Parvovirus, Paramyxovirus, Polyomavirus, Poxvirus, Rubella virus, Hepatitis virus, Reovirus (Rabies virus), Retrovirus, human immunodeficiency virus (HIV), Norovirus (Norwalk virus), Hemorrhagic fever virus, Mosquito and Tick-borne encephalitis virus, and Prions.

36. The method according to claim 35, wherein the antigenic protein or peptide is from a Filovirus selected from the group consisting of Ebola Virus and Marburg virus.

37. The method according to claim 32, wherein the antigenic protein or peptide is from a parasitic organism selected from the group consisting of Acanthamoeba species, Babesia species, Cryptosporidium species, Entamoeba species, Giardia species, Leishmania species, Naegleria species, Plasmodium species, Toxoplasma species, Trichomonas species, and Trypanosoma species.

38. The method according to claim 32, wherein the antigenic protein or peptide is a protein or peptide derived from the matrix 2 protein ectodomain of Influenza virus (M2e4×Het).

39. The method according to claim 29, wherein the polymeric coating is a polymer selected from the group consisting of polyesters, polyamides, polyphosphazines, polypropyl fumarates, poly(amino acids), polyethers, polyacetals, polycyanoacrylates, polyurethanes, polycarbonates, polyanhydrides, poly(ortho esters), polyhydroxy acids, polyacrylates, polymethacrylates, polyethylene-vinyl acetates, cellulose acetate polymers, polystyrenes, poly(vinyl chloride), poly(vinyl fluoride), poly(vinyl imidazole), poly(vinyl alcohol), water insoluble proteins, crosslinked proteins, aggregated proteins, water insoluble polysaccharides, crosslinked polysaccharides, aggregated polysaccharides, water insoluble polynucleotides, crosslinked polynucleotides, aggregated polynucleotides, water insoluble lipids and adducts thereof, crosslinked lipids and adducts thereof, and aggregated lipids and adducts thereof.

40. The method according to claim 39, wherein the polymeric coating is a polymer selected from the group consisting of poly(lactic-co-glycolic acid) (PLGA), polycaprolactone, polyglycolide, polylactic acid, and poly-3-hydroxybutyrate.

41. The method according to claim 40, wherein the polymeric coating is PLGA.

42. The method according to claim 29, wherein a plurality of fusion proteins are displayed on a plurality of cell vesicles.


SEQUENCE LISTING (1 sequence)

SEQ ID NO: 1 - protein, 23 aa, Influenza A virus
SLLTEVETPIRNEWGCRCNDSSD